(19) United States
(12) Patent Application Publication
(10) Pub. No.: US 2020/0046625 A1
(43) Pub. Date: Feb. 13, 2020

(54) COMPOUNDS USEFUL FOR THE TREATMENT AND/OR CARE OF THE SKIN, HAIR, NAILS AND/OR MUCOUS MEMBRANES

(71) Applicant: LUBRIZOL ADVANCED MATERIALS, INC., Cleveland, OH (US)

(72) Inventors: Wim Van Den Nest, Gava, Barcelona (ES); Nuria Almiñana Doménech, Gava, Barcelona (ES); Consuelo Garcia, Gava, Barcelona (ES); Antonio Vicente Ferrer Montiel, Alicante (ES)

(73) Assignee: LUBRIZOL ADVANCED MATERIALS, INC., Cleveland, OH (US)

(21) Appl. No.: 16/341,338

(22) PCT Filed: Oct. 12, 2017
(86) PCT No.: PCT/US2017/056300

§ 371 Date: Apr. 11, 2019

(30) Foreign Application Priority Data
Oct. 13, 2016 (EP) 16382466.7

(51) Int. Cl. A61K 8/64; C07K 5/087; A61K 38/06; A61Q 19/08; A61K 8/34; A61K 8/73; A61K 8/24; A61K 8/362

(52) U.S. Cl. CPC A61K 8/64; C07K 5/0812; A61K 38/06; A61Q 19/08; A61K 8/362; A61K 8/345; A61K 8/732; A61K 8/735; A61K 8/24; A61K 8/347

(57) ABSTRACT

A peptide has the formula (I) R₁—Wₘ—Xₙ-AA₁-AA₂-AA₃-Yₚ—Z_{q}—R₂, where AA₁ is Tyr or Trp, AA₂ is Val, Ile, Leu or Met, AA₃ is Tyr, Phe or Trp, W, X, Y and Z are amino acids, m, n, p and q are each 0 or 1, and m+n+p+q is less than or equal to 2. R₁ is H, a polymer derived from polyethylene glycol, a non-cyclic aliphatic group, alicyclyl, heterocyclyl, heteroarylalkyl, aryl, aralkyl, or R₅—CO—. R₅ is H, a non-cyclic aliphatic group, alicyclyl, aryl, aralkyl, heterocyclyl or heteroarylalkyl. R₂ is —NR₃R₄, —OR₃, or —SR₃. R₃ and R₄ are each H, a polymer derived from polyethylene glycol, a non-cyclic aliphatic group, alicyclyl, heterocyclyl, heteroarylalkyl, aryl, or aralkyl. R₁ and R₂ are not amino acids. Cosmetic or pharmaceutical compositions may include the peptide. It find use in reduction of lipid accumulation in the skin, treatment of cellulite, and treatment of symptoms of skin aging.

This application claims the priority of International Application PCT/US2017/056300, filed Oct. 12, 2017, and EP 16382446.7, filed Oct. 13, 2016, from which the PCT application claims priority, the disclosures of which are incorporated herein by reference, in their entireties.

### FIELD OF THE INVENTION

The invention relates to compounds effective in reducing lipid accumulation in the skin and to cosmetic and pharmaceutical compositions comprising the compounds. The compounds are useful in therapeutic and non-therapeutic treatments and/or care of the skin, hair, nails and/or mucous membranes. In particular, the compounds are useful for improving the appearance of skin affected by cellulite.

### BACKGROUND OF THE INVENTION

In humans, as well as other animal species, a large portion of social behavior and physiological function varies from day to night in a rhythmic fashion. The system that defines the circadian clock comprises central and peripheral components. In mammals, the central component of this oscillatory system resides in the suprachiasmatic nucleus (SCN) of the anterior hypothalamus [Boivin D. B. et al., “Circadian clock genes oscillate in human peripheral blood mononuclear cells”, Blood 2003, December, 102(12), 4143-4145]. This nucleus mainly functions by reception of light signals from specialized retina cells, the retina ganglion cells, and by activating a series of transcriptional, translational, and posttranslational mechanisms involving several genes, such as CLOCK, BMAL1, PER, and CRY that result in cascades of gene expression with 24 hour periodicity [Green, C. B., et al., “The Meter of Metabolism”, Cell, 2008, 134(5), 728-742]. Peripheral tissues and organs also possess autonomous regulatory systems that are independent of the central clock, but use the same machinery of genes and show entrainment to external stimuli that the tissues and organs may be subject to [Kawai M. and Rosen C. J., “PPARγ: a circadian transcription factor in adipogenesis and osteogenesis”, Nat. Rev. Endocrinol., 2010, 6, 629-636].

Some functions that intervene in the regulation of circadian rhythms, or which are subject to them, are the production of hormones, cytokine levels, temperature regulation or glucose levels, amongst others [Mehling, A., and Fluhr, J. W., “Chronobiology: biological clocks and rhythms of the skin”, Skin Pharmacol. Physiol., 2006, 19(4), 182-9].

Several of the skin's functions are also subject to circadian rhythms. It has been observed that various parameters investigated in women, such as blood circulation, skin amino acid content and transepidermal water loss (TEWL) increase during the night. On the other hand, the production of sebum, measured on the forehead using a sebumeter, gives its highest values around midday. The pH of the skin tends to decrease throughout the night and then increase throughout the day. The skin's blood circulation exhibits circadian rhythms and ranges during the day from low circulation early morning to then increasing, reaching its maximum values in the final hours of the afternoon going on to evening. Interestingly, circadian rhythms can also be found at cellular level in the skin, for example, it has been observed that proliferation of epidermal cells demonstrates its highest values at around 11 pm. [Mehling A. and Fluhr J. W., “Chronobiology: biological clocks and rhythms of the skin”, Skin Pharmacol. Physiol, 2006, 19(4), 182-9].

The skin is a physical barrier between an organism and their environment. The skin is composed of two tissues: the epidermis and the dermis. In the skin, the adipocytes are located in the deepest layer of the dermis, the hypodermis. The adipocytes are organized in lobules, separated by septa of connective tissue that contain vessels, nerves and lymph nodes. The main function of the adipocytes is the storage of fat in vacuoles in the form of triglycerides. In addition to this energy-related function, these cells are also involved in the production of some hormones as well as in the synthesis of molecules implicated in inflammatory response.

A condition that is related to the adipose cells (i.e. adipocytes) of the hypodermis, and which has been highly focused on by the cosmetic industry, is cellulite. Cellulite is the result of an excessive accumulation of lipids in the adipose tissue which puts a considerable amount of pressure on the surrounding epithelial tissue, resulting in an irregular appearance of the skin with the presence of dimples. This “orange peel” appearance of the skin is undesirable from an aesthetic point of view.

Cellulite has also been associated with the early onset of skin aging characteristics such as altered dermis properties. These properties include premature alteration of biomechanical properties of the skin such as viscoelastic properties measured in the form of retractability and elasticity indices using a Dermal Torque Meter®. It has been found that cellulite can have an impact on skin aging with a study finding that the population with cellulite present skin aging characteristics at an earlier age [J. P. Ortonne, et al., “Cellulite and skin ageing: is there any interaction?”, Journal of the European Academy of dermatology and Venereology 2008, 22, 827-834]. The treatment or prevention of skin aging, in particular premature skin aging due to the presence of cellulite, is desirable from an aesthetic point of view.

For the treatment of cellulite, a number of agents exist that stimulate reduction of the volume of adipose tissue by eliminating water, thus showing a draining effect. Furthermore, other agents with firming effect in the treatment of cellulite can also be used that correct the irregular appearance of the skin.

A widely used anti-cellulite agent is caffeine due to its lipolytic effect on adipocytes [Vogelgesang, B., et al., “In vitro and in vivo efficacy of sulfo-carrabiose, a sugar-based cosmetic ingredient with anti-cellulite properties”, Int. J. Cosmet. Sci., 2011, 33(2), 120-5], in addition to its draining effects. Furthermore, a high number of alternative agents also exist that possess similar mechanisms. A recent strategy in the search for new anti-cellulite agents is based on the influence over the actions related to circadian rhythms in the skin, [Dupressoir, A. et al., “Characterization of a mammalian gene related to the yeast CCR4 general transcription factor and revealed by transposon insertion”, J. Biol. Chem. 1999, 274(43), 31068-75; Dupressoir, A., et al., “Identification of four families of y CCR⁴⁻ and Mg²⁺-dependent endonuclease-related proteins in higher eukaryotes, and characterization of orthologs of y CCR4 with a conserved leucine-rich repeat essential for hCAF1/hPOP2 binding”, BMC Genomics, 2001, 2:9; Green, C. B., et al., “Loss of Nocturnin, a circadian deadenylase, confers resistance to hepatic steatosis and diet-induced obesity”, Proc. Nat. Acad. Sci. USA, 2007, 104(23), 9888-93] which similar to other tissues, experiences functional variations due to changes between day and night.

Other aesthetically undesirable conditions associated with the accumulation of lipids/aging include the development and presence of lower eyelid bags (also known as eyebags). Prolapse of orbital fat, an effect associated with advancing age, is a contributing factor to lower eyelid bags [R. A. Goldberg, et al., “What Causes Eyelid Bags? Analysis of 114 Consecutive Patients”, Plastic and Reconstructive Surgery, Apr. 15, 2005, pages 1395 to 1402].

One of the nocturnal functions in which an effect has been observed is adipogenesis (i.e. adipocyte maturation from precursor cells); one of the genes that has been studied which has an effect on adipogenesis is nocturnin. In some of the first studies carried out with Xenopus laevis retina that aimed to isolate genes affected by circadian rhythms, it was observed that the expression nocturnin mRNA was present at high levels in early night [Green, C. B., and Besharse, J. C., “Identification of a novel vertebrate circadian clock-regulated gene encoding the protein Nocturnin”, Proc. Nat. Acad. Sci. USA, 1996, 93(25), 14884-8]. It was later found that, in addition to being expressed in the retina in rats, nocturnin mRNA was expressed in numerous tissues, such as the liver, brain, lung, heart, ovary, skeletal muscle, testicles and bone marrow. Similarly, it was observed that a great circadian variation existed in the expression of nocturnin mRNA, with its maximum levels occurring at the beginning of the night [Dupressoir, A., et al., “Characterization of a mammalian gene related to the yeast CCR4 general transcription factor and revealed by transposon insertion”, J. Biol. Chem. 1999, 274(43), 31068-75; Dupressoir, A., et al., “Identification of four families of yCCR⁴⁻ and Mg²⁺-dependent endonuclease-related proteins in higher eukaryotes, and characterization of orthologs of yCCR4 with a conserved leucine-rich repeat essential for hCAF1/hPOP2 binding”, BMC Genomics, 2001, 2:9].

Regarding the function of nocturnin, studies with knockout mice that were unable to express nocturnin demonstrated that, even though these individuals showed normal general circadian behavior, they presented resistance to diet-induced obesity and they also showed other metabolic changes such as a lower accumulation of lipids in the liver. Therefore, this metabolic phenotype suggests that nocturnin controls specific secondary circadian channels associated with the accumulation and use of lipids [Green C. B. et al., “Loss of Nocturnin, a circadian deadenylase, confers resistance to hepatic steatosis and diet-induced obesity”, Proc. Nat. Acad. Sci. USA, 2007, 104(23), 9888-93]. Another one of the functions in which nocturnin's involvement has been observed is inflammation. Nocturnin stabilizes the pro-inflammatory transcript iNOS and a reduction in nocturnin would mean a reduction in inflammation. [Niu, S., et al., “The circadian deadenylase Nocturnin is necessary for stabilization of the iNOS mRNA in mice” PloS one 2011, 6(11), e26954].

As nocturnin is a marker that is correlated with the accumulation and use of lipids, it may be used as a base for further studies to identify stimulation or reduction activity on the accumulation of lipids in adipocytes in the dermis and in the treatment of inflammation.

There is a need for a new active agent that is effective at treating and/or preventing conditions associated with the accumulation of lipids in the skin. In particular, there is a need for a new active agent that is effective in reducing the accumulation of lipids in the skin; useful in the treatment and/or prevention of cellulite; useful in reducing and/or preventing the symptoms of skin aging, and/or useful in the treatment of inflammation of the skin.

The present invention sets out to solve some or all of the above-identified problems and meet some or all of the above-identified needs.

### SUMMARY OF THE INVENTION

In a first aspect, the invention provides a compound represented by formula (I):

R₁—Wₘ—Xₙ-AA₁-AA₂-AA₃-Yₚ—Z_{q}—R₂ (I),

its stereoisomers and/or its cosmetically or pharmaceutically acceptable salts, wherein:
AA₁ is selected from the group consisting of Tyr and Trp;
AA₂ is selected from the group consisting of Val, Ile, Leu and Met;
AA₃ is selected from the group consisting of Tyr, Phe and Trp;
W, X, Y and Z are each independently an amino acid;
m, n, p and q are each independently 0 or 1;
m+n+p+q is less than or equal to 2;
R₁ is selected from the group consisting of H, a polymer derived from polyethylene glycol, a non-cyclic aliphatic group, alicyclyl, heterocyclyl, heteroarylalkyl, aryl, aralkyl and R₅—CO—, wherein R₅ is selected from the group consisting of H, a non-cyclic aliphatic group, alicyclyl, aryl, arylalkyl, heterocyclyl, and heteroarylalkyl;
R₂ is selected from the group consisting of —NR₃R₄, —OR₃, and —SR₃, wherein R₃ and R₄ are independently selected from a group consisting of H, a polymer derived from polyethylene glycol, a non-cyclic aliphatic group, alicyclyl, heterocyclyl, heteroarylalkyl, aryl and aralkyl; and
R₁ and R₂ are not amino acids.

The present invention is based on the finding that compounds of formula (I) are effective in reducing the levels of nocturnin in subcutaneous pre-adipocyte cells overnight and in reducing lipid accumulation in subcutaneous pre-adipocyte cells, and that compounds of formula (I) can be used in the treatment of the skin, hair, nails and/or mucous membranes. In particular, the compounds of the invention are effective in non-therapeutic and therapeutic treatments of conditions of the skin, hair, nails and/or mucous membranes that are associated with the presence of nocturnin, the accumulation of lipids and/or the function of adipocytes.

In another aspect, the invention provides a cosmetic or pharmaceutical composition comprising a compound of formula (I), its stereoisomers and/or its cosmetically or pharmaceutically acceptable salts, together with at least one cosmetically or pharmaceutically acceptable excipient or adjuvant.

In another aspect, the invention provides the use of a compound of formula (I), its stereoisomers and/or its cosmetically acceptable salts, or a cosmetic composition comprising a compound of formula (I), its stereoisomers and/or its cosmetically acceptable salts, for the cosmetic, non-therapeutic treatment and/or care of the skin, hair, nails and/or mucous membranes. In particular, the invention relates to use of the compound of the invention in the reduction of lipid accumulation in the skin, the treatment of cellulite and/or the treatment of the symptoms of skin aging.

In another aspect, the invention provides a cosmetic, non-therapeutic method of treatment and/or care of the skin, hair, nails and/or mucous membranes in a subject comprising administering a cosmetically effective amount of a compound of formula (I), its stereoisomers and/or its cosmetically acceptable salts, or a cosmetic composition comprising same, to the subject. Typically, the compound will be administered topically, and, in particular for the treatment of cellulite, preferably the compound is administered at night.

In another aspect, the invention provides a compound of formula (I), its stereoisomers and/or its pharmaceutically acceptable salts, or a pharmaceutical composition comprising same, for use as a medicament. In particular, the invention provides a compound of formula (I), its stereoisomers and/or its pharmaceutically acceptable salts, or a pharmaceutical composition comprising same, for use in the treatment or prevention of a disease or disorder which is associated with the presence of nocturnin, the accumulation of lipids and/or the function of adipocytes in the skin. In particular, the invention relates to the compound of formula (I), its stereoisomers and/or its pharmaceutically acceptable salts, or a pharmaceutical composition comprising same, for use in the treatment of inflammation of the skin.

In another aspect, the invention provides for the use of the compound of formula (I), its stereoisomers and/or its pharmaceutically acceptable salts for the manufacture of a medicament for the treatment or prevention of a disease or disorder. The disease or disorder is associated with the presence of nocturnin, the accumulation of lipids and/or the function of adipocytes in the skin, such as inflammation of the skin.

In another aspect, the invention provides a method of treating or preventing a disease or disorder in a subject comprising administering a therapeutically effective amount of a compound of formula (I) or a pharmaceutical composition comprising same, to the subject. The disease or disorder is associated with the presence of nocturnin, the accumulation of lipids and/or the function of adipocytes in the skin, such as inflammation of the skin.

### DETAILED DESCRIPTION OF THE INVENTION

### Definitions

In the context of this invention “skin” is understood to be the layers which comprise it, from the uppermost layer or stratum corneum to the lowermost layer or hypodermis, both inclusive. These layers are composed of different types of cells such as keratinocytes, fibroblasts, melanocytes, mast cells, neurones and/or adipocytes among others. The term “skin” also comprises the scalp. The term “skin” includes the skin of mammals and includes human skin. Likewise, the terms “hair, nails and mucous membranes” include the hair, nails and mucous membranes of mammals, for example humans.

The term “treatment” covers therapeutic methods including methods directed to the administration of a compound according to the invention to alleviate or eliminate a disease or disorder, or to reduce or eliminate one or more symptoms associated with said disease or disorder. The term “treatment” also covers methods of therapy directed to alleviating or eliminating physiological consequences of the disease or disorder.

When the terms “treatment” and “care” are accompanied by the qualifications “cosmetic” and/or “non-therapeutic”, it means that the treatment or care is such and, for example, has the aim of improving or maintaining the aesthetic appearance of the skin, hair, nails and/or mucous membranes. In particular, the treatment can have the aim of improving cosmetic properties of the skin, hair, nails and/or mucous membranes such as, for example and not restricted to, the level of hydration, elasticity, firmness, shine, tone or texture, which properties affect the aesthetic appearance of the skin, hair, nails and/or mucous membranes. The term “care” in the context of this specification refers to the maintenance of properties of the skin, hair, nails and/or mucous membranes. Said properties are subject to being improved or maintained by cosmetic treatment and/or care of the skin, hair, nails and/or mucous membranes both in healthy subjects (for example, in the cosmetic, non-therapeutic treatment and/or care of skin which is not inflamed, such as when the subject does not suffer inflammation of the skin) as well as in those which present diseases and/or disorders of the skin, hair, nails and/or mucous membranes such as, for example and not restricted to, ulcers and injuries to skin, psoriasis, dermatitis, acne or rosacea, among others.

The term “prevention”, as used in this invention, refers to the ability of a compound of the invention to prevent, delay or hinder the appearance or development of a disease or disorder, or to prevent, delay or hinder the change in a cosmetic property of the skin, mucous membranes and/or hair. The term “prevention”, as used in this invention, is interchangeable with the term “inhibition”, i.e., it refers to the ability of a compound of the invention to inhibit the appearance or development of a disease or disorder, or to inhibit the change in a cosmetic property of the skin, hair, nails and/or mucous membranes.

In the context of this invention, the term “aging” refers to the changes experienced by the skin with age (chronoaging) or through exposure to the sun (photoaging) or to environmental agents such as tobacco smoke, extreme climatic conditions of cold or wind, chemical contaminants or pollutants, and includes all the external visible and/or perceptible changes through touch, such as and not restricted to, the development of discontinuities on the skin such as wrinkles, fine lines, expression lines, stretch marks, furrows, irregularities or roughness, increase in the size of pores, loss of hydration, loss of elasticity, loss of firmness, loss of smoothness, loss of the capacity to recover from deformation, loss of resilience, sagging of the skin such as sagging cheeks, the appearance of bags under the eyes or the appearance of a double chin, among others, changes to the color of the skin such as marks, reddening, bags or the appearance of hyperpigmented areas such as age spots or freckles among others, anomalous differentiation, hyperkeratinization, elastosis, keratosis, hair loss, orange-peel skin, loss of collagen structure and other histological changes of the stratum corneum, of the dermis, epidermis, vascular system (for example the appearance of spider veins or telangiectasias) or of those tissues close to the skin, among others. The term “photoaging” groups together the set of processes due to the prolonged exposure of the skin to ultraviolet radiation which result in the premature aging of the skin, and it presents the same physical characteristics as aging, such as and not restricted to, flaccidity, sagging, changes to the color or irregularities in the pigmentation, abnormal and/or excessive keratinization. The sum of various environmental factors such as exposure to tobacco smoke, exposure to pollution, and climatic conditions such as cold and/or wind also contribute to the aging of the skin.

“Body Mass Index” (BMI), also known as Quetelet index, is a simple index of weight-for-height that is used to classify underweight, overweight and obesity in adults. It is calculated by dividing body weight (in kg) by the square of the body height (in m²). Commonly accepted ranges of BMI values include 18.5 to 24.99 kg/m² for a subject of normal weight and greater than or equal to 25.00 kg/m² for an overweight subject is greater than or equal to 25.00 kg/m² (values from the World Health Organization). Ideal body weight will vary among species and individuals based on age, height, body build, bone structure and sex.

In this description the abbreviations used for amino acids follow the rules of IUPAC-IUB Commission of Biochemical Nomenclature specified in Eur. J. Biochem., (1984), 138, 9-37. Thus, for example, Gly represents NH₂—CH₂—COOH, Gly- represents NH₂—CH₂—CO—, -Gly represents —NH—CH₂—COOH and -Gly- represents —NH—CH₂—CO—. Therefore, the hyphen, which represents the peptide bond, eliminates the OH in the 1-carboxyl group of the amino acid (represented here in the conventional non-ionized form) when situated to the right of the symbol, and eliminates the H of the 2-amino group of the amino acid when situated to the left of the symbol; both modifications can be applied to the same symbol (see Table 1).

TABLE 1
Structures of the amino acid residues, their nomenclature in three-letter code and nomenclature
for the amino acids in one letter code

| Name | Residue | Symbol | Residue |
|---|---|---|---|
| Tyrosyl -Tyr- Y | 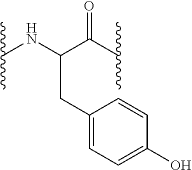 [SMILES: *NC(Cc1ccc(O)cc1)C(*)=O] | Phenylalanyl -Phe- F | 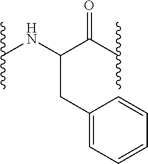 [SMILES: *NC(Cc1ccccc1)C(*)=O] |

| | | | |
|---|---|---|---|
| Tryptophyl -Trp- W | 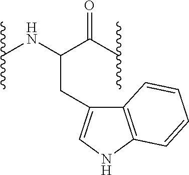 [SMILES: *NC(Cc1c[nH]c2ccccc12)C(*)=O] | Valyl -Val- V | 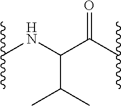 [SMILES: *NC(C(*)=O)C(C)C] |

| | | | |
|---|---|---|---|
| Isoleucyl -Ile- I | 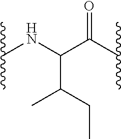 [SMILES: *NC(C(*)=O)C(C)CC] | Leucyl -Leu- L | 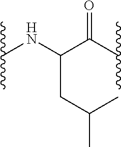 [SMILES: *NC(CC(C)C)C(*)=O] |
| Methionyl -Met- M | 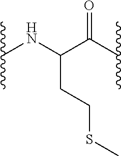 [SMILES: *NC(CCSC)C(*)=O] | | |

As used herein, the term “non-cyclic aliphatic group” includes linear (i.e., straight and unbranched) or branched, saturated or unsaturated hydrocarbyl groups such as alkyl, alkenyl and alkynyl. The non-cyclic aliphatic group may be substituted (mono- or poly-) or unsubstituted.

As used herein, the term “alkyl” includes both saturated linear and branched alkyl groups, which may be substituted (mono- or poly-) or unsubstituted. The alkyl group is bound to the rest of the molecule by a single bond. The alkyl group has from 1 to 24, preferably from 1 to 16, more preferably from 1 to 14, even more preferably from 1 to 12, yet more preferably 1, 2, 3, 4, 5 or 6 carbon atoms. The term “alkyl” includes, for example, methyl, ethyl, isopropyl, isobutyl, tert-butyl, 2-methylbutyl, heptyl, 5-methylhexyl, 2-ethylhexyl, octyl, decyl, dodecyl, lauryl, hexadecyl, octadecyl and amyl.

As used herein, the term “alkenyl” refers to a group containing one or more double carbon-carbon bonds and which may be linear or branched and substituted (mono- or poly-) or unsubstituted. Preferably it has 1, 2 or 3 double carbon-carbon bonds. If more than one double carbon-carbon bond is present, the double bonds may be conjugated or not conjugated. Preferably the alkenyl group has from 2 to 24, preferably from 2 to 16, more preferably from 2 to 14, even more preferably from 2 to 12, yet more preferably 2, 3, 4, 5 or 6 carbon atoms. The alkenyl group is bound to the rest of the molecule by a single bond. The term “alkenyl” includes, for example, vinyl (—CH₂═CH₂), allyl (—CH₂—CH═CH₂), prenyl, oleyl, linoleyl groups and similar.

The term “alkynyl” refers to a group containing one or more triple carbon-carbon bonds and which may be linear or branched, and substituted (mono- or poly-) or unsubstituted. Preferably the alkynyl group has 1, 2 or 3 triple carbon-carbon bonds. The triple bonds may be conjugated or not conjugated. The alkynyl group has from 2 to 24, preferably from 2 to 16, more preferably from 2 to 14, even more preferably from 2 to 12, yet more preferably 2, 3, 4, 5 or 6 carbon atoms. The alkynyl group is bound to the rest of the molecule by a single bond. The term “alkynyl” includes, for example and not restricted to, ethynyl, 1-propynyl, 2-propynyl, 1-butynyl, 2-butynyl, 3-butynyl, pentynyl, such as 1-pentynyl, and similar. The alkynyl group can also contain one or more double carbon-carbon bonds, and alkynyl groups include, for example and not restricted to, but-1-en-3-ynyl and pent-4-en-1-ynyl groups, and similar.

The term “alicyclyl” is used herein to cover, for example and not restricted to, aliphatic cyclic (alicyclic) groups such as cycloalkyl or cycloalkenyl or cycloalkynyl groups. The term “alicyclyl” refers to a monoradical that contains one or more rings of carbon atoms, the rings may be saturated (e.g., cyclohexyl) or unsaturated (e.g., cyclohexenyl) provided that they are not aromatic. More specifically alicylic groups contain three or more, from 3 to 24, from 3 to 12, or from 6 to 12, ring carbon atoms. The alicyclic group may be a monocyclic, bicyclic, or tricyclic ring system and the rings may be, for example, fused or linked by a single bond or a linking group such as a methylene or other alkylene group. The alicyclic group may be substituted (mono- or poly-) or unsubstituted. In one embodiment, the alicyclyl group is a 6 to 12 membered ring system which consists of carbon atoms and optionally contains one or two double bonds.

The term “cycloalkyl” refers to a saturated mono- or polycyclic alkyl group which may be substituted (mono- or poly-) or unsubstituted. The cycloalkyl group has from 3 to 24, preferably from 3 to 16, more preferably from 3 to 14, even more preferably from 3 to 12, yet even more preferably 3, 4, 5 or 6 carbon atoms. The cycloalkyl group is bound to the rest of the molecule by a single bond, Cycloalkyl groups include, for example and not restricted to, cyclopropyl, cyclobutyl, cyclopentyl, cyclohexyl, cycloheptyl, methyl cyclohexyl, dimethyl cyclohexyl, octahydroindene, decahydronaphthalene, dodecahydrophenalene and similar.

The term “cycloalkenyl” refers to a non-aromatic mono- or polycyclic alkenyl group which may be substituted (mono- or poly-) or unsubstituted. The cycloalkenyl group has from 5 to 24, preferably from 5 to 16, more preferably from 5 to 14, even more preferably from 5 to 12, yet more preferably 5 or 6 carbon atoms. The cycloalkenyl group is bound to the rest of the molecule by a single bond. Preferably the cycloalkenyl group contains 1, 2 or 3 double carbon-carbon bonds. If more than one double carbon-carbon bond is present, the double bonds may be conjugated or not conjugated. Cycloalkenyl groups include, for example and not restricted to, the cyclopent-1-en-1-yl group and similar.

The term “cycloalkynyl” refers to a non-aromatic mono- or polycyclic alkynyl group which may be substituted (mono- or poly-) or unsubstituted. The cycloalkynyl group has from 8 to 24, preferably from 8 to 16, more preferably from 8 to 14, even more preferably from 8 to 12, yet even more preferably 8 or 9 carbon atoms and is bound to the rest of the molecule by a single bond. Preferably the cycloalkynyl group contains 1, 2 or 3 triple carbon-carbon bonds, conjugated or not conjugated. Cycloalkynyl groups include, for example and not restricted to, the cyclooct-2-yn-1-yl group and similar. Cycloalkynyl groups can also contain one or more double carbon-carbon bonds, including, for example and not restricted to, the cyclooct-4-en-2-ynyl group and similar.

As used herein, the term “heterocyclyl” or “heterocyclic” refers to a hydrocarbon ring system of 3 to 10 members, wherein one or more of the atoms in the ring or rings is a heteroatom (i.e. not a carbon atom). Thus “heterocyclyl” or “heterocyclic” refers a cyclic group in which the ring atoms consist of carbon and one or more heteroatoms. To satisfy valence, the heteroatom may be bonded to H or substituent groups. Preferably from 1, 2 or 3 of the ring carbon atoms are heteroatoms. Each heteroatom can be independently selected from the group consisting of O, N, S, P and B, or the group consisting of O, N, and S. The heterocyclyl group may be substituted (mono- or poly-) or unsubstituted. The heterocyclyl group may be a monocyclic, bicyclic, or tricyclic ring system and the rings may be, for example, fused or linked by a single bond or a linking group such as a methylene or other alkylene group. Nitrogen, carbon or sulfur atoms present in the heterocyclyl radical may be optionally oxidized and the nitrogen atom may be optionally quaternized. The heterocyclyl radical may be unsaturated or partially or fully saturated. The heterocyclyl radical may be aliphatic or aromatic. In one embodiment, the heterocyclyl is aliphatic (also known as heteroalicyclyl) and is a 3 to 10 membered ring system where the atoms of the ring or rings consist of carbon atoms and from 1 to 4, or 1, 2 or 3 heteroatoms. In one embodiment, the heterocyclyl group is a 6 to 10 membered ring system where the atoms of the ring or rings consist of carbon atoms and from 1 to 4 heteroatoms and where the ring system optionally contains one or two double bonds. In one embodiment, the heterocyclyl is aromatic (also known as heteroaryl) and is a 6 to 10 membered ring system where the atoms of the ring or rings consist of carbon atoms and from 1 to 4, or 1, 2 or 3 heteroatoms. The greatest preference is for the term heterocyclyl to refer to a ring of 5 or 6 members. Examples of saturated heteroalicyclyl groups are dioxane, piperidine, piperazine, pyrrolidine, morpholine and thiomorpholine. Examples of aromatic heterocyclyl groups are pyridine, pyrrole, furan, thiophene, benzofuran, imidazoline, quinolein, quinoline, pyridazine and naphthyridine.

The term “aryl group” refers to an aromatic group which has from 6 to 30, preferably from 6 to 18, more preferably between 6 and 10, yet even more preferably 6 or 10 carbon atoms. The aryl group can comprise 1, 2, 3 or 4 aromatic rings, which may be linked by a carbon-carbon bond or fused together and includes, for example and not restricted to, phenyl, naphthyl, diphenyl, indenyl, phenanthryl or antranyl among others. The aryl group may be substituted (mono- or poly-) or unsubstituted.

The term “aralkyl group” refers to an alkyl group substituted by an aromatic group, with from 7 to 24 carbon atoms and including, for example and not restricted to, —(CH₂)₁₋₆-phenyl, —(CH₂)₁₋₆-(1-naphthyl), —(CH₂)₁₋₆-(2-naphthyl), —(CH₂)₁₋₆—CH(phenyl)₂ and similar.

The term “heteroarylalkyl” refers to an alkyl group substituted by a heteroaryl (also known as aromatic heterocyclic) group as defined above, the alkyl group having from 1 to 6 carbon atoms and the heteroaryl group having from 2 to 24 carbon atoms and from 1 to 3 heteroatoms. Heteroarylalkyl groups include, for example and not restricted to, —(CH₂)₁₋₆-imidazolyl, —(CH₂)₁₋₆-triazolyl, —(CH₂)₁₋₆-thienyl, —(CH₂)₁₋₆-furyl, —(CH₂)₁₋₆-pyrrolidinyl and similar.

As is understood in this technical field, there may be a certain degree of substitution of the aforementioned groups. In particular, there can be substitution in any of the groups identified above where it is explicitly stated. The substituted groups (radicals) referred to above are groups (or radicals) which are substituted in one or more positions available by one or more substituents. Preferably substitution is in the 1, 2 or 3 positions, more preferably in the 1 or 2 positions, yet even more preferably in the 1 position. Suitable substituents include, for example and not restricted to: C₁-C₄ alkyl; hydroxyl; C₁-C₄ alkoxyl; amino; amino-C₁-C₄ alkyl; C₁-C₄ carbonyloxyl; C₁-C₄ oxycarbonyl; halogen such as fluoride, chlorine, bromine and iodine; cyano; nitro; azide; C₁-C₄ alkylsulfonyl; thiol; C₁-C₄ alkylthio; aryloxy such as phenoxyl; —NR_{b}(C═NR_{b})NR_{b}R_{c}; wherein R_{b} and R_{c} are independently selected from the group consisting of H, C₁-C₄ alkyl, C₂-C₄ alkenyl, alkynyl, C₃-C₁₀ cycloalkyl, C₆-C₁₈ aryl, C₇-C₁₇ aralkyl, heterocyclyl of 3-10 members, and protective group of the amino group.

As used herein, the term “comprising”, which is inclusive or open-ended and does not exclude additional unrecited elements or method steps, is intended to encompass as alternative embodiments, the phrases “consisting essentially of” and “consisting of” where “consisting of” excludes any element or step not specified and “consisting essentially of” permits the inclusion of additional unrecited elements or steps that do not materially affect the essential or basic and novel characteristics of the composition or method under consideration.

### Compounds of the Invention

A first aspect of the invention relates to a compound of formula (I)

—R₁—Wₘ—Xₙ-AA₁-AA₂-AA₃-Yₚ—Z_{q}—R₂ (I),

its stereoisomers, mixtures thereof and/or cosmetically or pharmaceutically acceptable salts thereof, wherein:
AA₁ is selected from the group consisting of Tyr and Trp;
AA₂ is selected from the group consisting of Val, Ile, Leu and Met;
AA₃ is selected from the group consisting of Tyr, Phe and Trp;
W, X, Y and Z are each independently an amino acid;
m, n, p and q are each independently 0 or 1;
m+n+p+q is less than or equal to 2;
R₁ is selected from the group consisting of H, a polymer derived from polyethylene glycol, a non-cyclic aliphatic group, alicyclyl, heterocyclyl, heteroarylalkyl, aryl, aralkyl and R₅—CO—, wherein R₅ is selected from the group consisting of H, a non-cyclic aliphatic group, alicyclyl, aryl, aralkyl, heterocyclyl and heteroarylalkyl;
R₂ is selected from the group consisting of —NR₃R₄, —OR₃, and —SR₃, wherein R₃ and R₄ are independently selected from a group consisting of H, a polymer derived from polyethylene glycol, a non-cyclic aliphatic group, alicyclyl, heterocyclyl, heteroarylalkyl, aryl and aralkyl; and
R₁ and R₂ are not amino acids.

The compound of formula (I) is a peptide which comprises 3, 4 or 5 amino acids linked in a chain. R₁ in bound to the amino terminal end (N-terminal) of the peptide and R₂ is bound to the carboxy-terminal end (C-terminal) of the peptide.

R₁ can be selected from the group consisting of H, a polymer derived from polyethylene glycol with a molecular weight between 200 and 35000 Daltons and R₅—CO—, wherein R₅ is selected from the group consisting of C₁-C₂₄ alkyl, C₂-C₂₄ alkenyl, C₂-C₂₄ alkynyl, C₃-C₂₄ cycloalkyl, C₅-C₂₄ cycloalkenyl, C₈-C₂₄ cycloalkynyl, C₆-C₃₀ aryl, C₇-C₂₄ aralkyl, 3-10 membered heterocyclyl ring, and a heteroarylalkyl containing from 2 to 24 carbon atoms and from 1 to 3 heteroatoms, wherein the alkyl group has 1 to 6 carbon atoms. In one embodiment, R₁ is selected from the group consisting of H and R₅—CO—, wherein R₅ is selected from the group consisting of C₁-C₁₈ alkyl, C₂-C₂₄ alkenyl, and C₃-C₂₄ cycloalkyl or the group consisting of C₁-C₁₆ alkyl, C₂-C₁₈ alkenyl, and C₃-C₇ cycloalkyl. The R₅—CO— group includes alkanoyl groups such as acetyl (CH₃—CO—, which is abbreviated herein as “Ac-”), myristoyl (CH₃—(CH₂)₁₂—CO—, which is abbreviated herein as “Myr-”) and palmitoyl (CH₃—(CH₂)₁₄—CO—, which is abbreviated herein as “Palm-”). In one embodiment, R₁ is selected from the group consisting of H, acetyl, tert-butanoyl, prenyl, hexanoyl, 2-methylhexanoyl, cyclohexanecarboxyl, octanoyl, decanoyl, lauroyl, myristoyl, palmitoyl, stearoyl, oleoyl and linoleoyl. In one embodiment, R₁ is selected from the group consisting of H and R₅—CO—, wherein R₅ is selected from the group consisting of C₁-C₁₆ alkyl and C₂-C₁₈ alkenyl. In one embodiment, R₁ is selected from the group consisting of H, acetyl, myristoyl and palmitoyl.

R₂ can be selected from the group consisting of —NR₃R₄, —OR₃, and —SR₃, wherein R₃ and R₄ are independently selected from the group consisting of H, a polymer derived from polyethylene glycol, C₁-C₂₄ alkyl, C₂-C₂₄ alkenyl, C₂-C₂₄ alkynyl, C₃-C₂₄ cycloalkyl, C₅-C₂₄ cycloalkenyl, C₈-C₂₄ cycloalkynyl, C₆-C₃₀ aryl, C₇-C₂₄ aralkyl, 3-10 membered heterocyclyl ring, and heteroarylalkyl containing from 2 to 24 carbon atoms and from 1 to 3 heteroatoms, wherein the alkyl group has 1 to 6 carbon atoms. Optionally, R₃ and R₄ can be joined by a saturated or unsaturated carbon-carbon bond, forming a ring with the nitrogen atom. In one embodiment, R₂ is —NR₃R₄ or —OR₃. In one embodiment, R₃ and R₄ are independently selected from the group consisting of H, a polymer derived from polyethylene glycol with a molecular weight of between 200 and 35000 Daltons, methyl, ethyl, hexyl, dodecyl and hexadecyl. In one embodiment R₃ and R₄ are independently selected from the group consisting of H and C₁-C₁₆ alkyl. In one embodiment, R₂ is not OR₃ where R₃ is a methyl group, i.e., R₂ is not OCH₃. In one embodiment R₃ is H and R₄ is selected from the group consisting of H and C₁-C₁₆ alkyl, including methyl, ethyl, hexyl, dodecyl and hexadecyl. In accordance with one embodiment, R₂ is selected from the group consisting of —OH, —NH₂ and —NHR₄ where R₄ is C₁-C₁₆ alkyl. R₄ can be C₆ alkyl, i.e., —C₆H₁₃ or C₁₆ alkyl, i.e., —C₁₆H₃₃.

In one embodiment R₁ is selected from the group consisting of H and R₅—CO—, wherein R₅ is selected from the group consisting of C₁-C₁₈ alkyl, C₂-C₂₄ alkenyl, and C₃-C₂₄ cycloalkyl; and R₂ is —NR₃R₄ or —OR₃ wherein R₃ and R₄ are independently selected from the group consisting of H and C₁-C₁₆ alkyl. In an exemplary embodiment, R₃ is H and R₄ is selected from the group consisting of H and C₁-C₁₆ alkyl; for example, R₂ is selected from the group consisting of —OH, —NH₂ and —NHR₄, where R₄ is C₁-C₁₆ alkyl.

In one embodiment, R₁ is selected from the group consisting of H and acetyl, tert-butanoyl, prenyl, hexanoyl, 2-methylhexanoyl, cyclohexanecarboxyl, octanoyl, decanoyl, lauroyl, myristoyl, palmitoyl, stearoyl, oleoyl and linoleoyl; and R₂ is —NR₃R₄ or —OR₃ wherein R₃ and R₄ are independently selected from the group consisting of H and C₁-C₁₆ alkyl. In an exemplary embodiment, R₃ is H and R₄ is selected from the group consisting of H and C₁-C₁₆ alkyl; for example, R₂ is selected from the group consisting of —OH, —NH₂ and —NHR₄ where R₄ is C₁-C₁₆ alkyl.

In one embodiment, R₁ is selected from the group consisting of H and R₅—CO—, wherein R₅ is selected from the group consisting of C₁-C₁₆ alkyl and C₂-C₁₈ alkenyl; and R₂ is —NR₃R₄ or —OR₃ wherein R₃ and R₄ are independently selected from the group consisting of H and C₁-C₁₆ alkyl. In an exemplary embodiment R₃ is H and R₄ is selected from the group consisting of H and C₁-C₁₆ alkyl; for example, R₂ is selected from the group consisting of —OH, —NH₂ and —NHR₄ where R₄ is C₁-C₁₆ alkyl.

In one embodiment, R₁ is selected from the group consisting of H, acetyl, myristoyl and palmitoyl; and R₂ is —NR₃R₄ or —OR₃ wherein R₃ and R₄ are independently selected from the group consisting of H and C₁-C₁₆ alkyl. In an exemplary embodiment R₃ is H and R₄ is selected from the group consisting of H and C₁-C₁₆ alkyl; for example, R₂ is selected from the group consisting of —OH, —NH₂ and —NHR₄ where R₄ is C₁-C₁₆ alkyl.

In one embodiment, R₁ is selected from the group consisting of a substituted non-cyclic aliphatic group, substituted alicyclyl, substituted heterocyclyl, substituted heteroarylalkyl, substituted aryl, substituted aralkyl and R₅—CO—, wherein R₅ is selected from the group consisting of a substituted non-cyclic aliphatic group, substituted alicyclyl, substituted aryl, substituted aralkyl, substituted heterocyclyl and substituted heteroarylalkyl; and/or R₂ is —NR₃R₄, wherein at least one of R₃ and R₄ is selected from the group consisting of a substituted non-cyclic aliphatic group, substituted alicyclyl, substituted heterocyclyl, substituted heteroarylalkyl, substituted aryl and substituted aralkyl, or R₂ is —OR₃, or —SR₃, wherein R₃ is selected from the group consisting of a substituted non-cyclic aliphatic group, substituted alicyclyl, substituted heterocyclyl, substituted heteroarylalkyl, substituted aryl and substituted aralkyl.

In accordance with another particular embodiment the most preferred structures of the polymer derived from polyethylene glycol are the group (—CH₂H₂—O)ᵣ—H in which r is a number comprised between 4 and 795 and the group

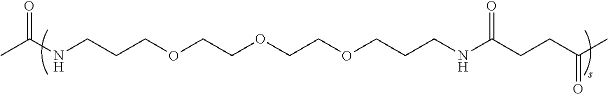

where s is a number between 1 and 125.

In one embodiment, the invention provides a compound of formula (I), wherein at least one of R₁ is not H; and R₂ is not OH.

The compound of the invention comprises a core group of the three amino acids AA₁, AA₂ and AA₃, linked together. AA₁ is selected from the group consisting of Tyr and Trp. AA₂ is selected from the group consisting of Val, Ile, Leu and Met. AA₃ is selected from the group consisting of Tyr, Phe and Trp. R₁ and R₂ are as defined above.

In one embodiment, the invention provides a compound of formula (I), wherein said compound is not H-Tyr-Val-Tyr-OCH₃.

In one embodiment, AA₁ is Tyr, AA₂ is selected from the group consisting of Val, Ile, Leu and Met, and AA₃ is selected from the group consisting of Tyr, Phe and Trp.

In one embodiment, AA₁ is Tyr, AA₂ is selected from the group consisting of Val, Ile, Leu and Met, and AA₃ is Tyr.

In one embodiment, AA₁ is Tyr, AA₂ is Val and AA₃ is Tyr. In an exemplary embodiment, AA₁ is Tyr, AA₂ is Val, AA₃ is Tyr, R₁ is selected from the group consisting of H, acetyl, myristoyl and palmitoyl, and R₂ is selected from the group consisting of —OH, —NH₂ and —NHR₄ where R₄ is C₁-C₁₆ alkyl. R₄ can be C₆ alkyl or C₁₆ alkyl. In an exemplary embodiment, AA₁ is Tyr, AA₂ is Val, AA₃ is Tyr, R₁ is H and R₂ is selected from the group consisting of —OH, —NH₂ and —NHR₄ where R₄ is C₁-C₁₆ alkyl. R₄ can be C₆ alkyl or C₁₆ alkyl. The compound of the invention can have the formula H-Tyr-Val-Tyr-NH₂. The compound of the invention can have the formula H-Tyr-Val-Tyr-OH. The compound of the invention can have the formula H-Tyr-Val-Tyr-NHC₆H₁₃. The compound of the invention can have the formula H-Tyr-Val-Tyr-NHC₁₆H₃₃. The compound of the invention can have the formula H-Tyr-Val-Tyr-Phe-NH₂. The compound of the invention can have the formula H-Tyr-Val-Tyr-Trp-NH₂. The compound of the invention can have the formula H-Phe-Tyr-Val-Tyr-Phe-NH₂. The compound of the invention can have the formula H-Phe-Trp-Tyr-Val-Tyr-NH₂. The compound of the invention can have the formula H-Tyr-Val-Tyr-Phe-Trp-NH₂. In an exemplary embodiment, AA₁ is Tyr, AA₂ is Val, AA₃ is Tyr, R₁ is selected from the group consisting of H, acetyl, myristoyl and palmitoyl, and R₂ is —NH₂. The compound of the invention can have the formula: Ac-Tyr-Val-Tyr-NH₂. The compound of the invention can have the formula Myr-Tyr-Val-Tyr-NH₂. The compound of the invention can have the formula Palm-Tyr-Val-Tyr-NH₂. In an exemplary embodiment, AA₁ is Tyr, AA₂ is Val, AA₃ is Tyr, R₁ is selected from the group consisting of acetyl, myristoyl and palmitoyl, and R₂ is —NH₂.

When, AA₁ is Tyr, AA₂ is Val, AA₃ is Tyr, for example, as in the above-mentioned embodiments, the sum of m, n, p and q can be 0.

When, AA₁ is Tyr, AA₂ is Val, AA₃ is Tyr, for example, as in the above-mentioned embodiments, the sum of m, n, p and q can be 1 and W, X, Y and Z are each independently selected from the group consisting of Phe and Trp.

When, AA₁ is Tyr, AA₂ is Val, AA₃ is Tyr, for example, as in the above-mentioned embodiments, the sum of m, n, p and q can be 2 and W, X, Y and Z are each independently selected from the group consisting of Phe and Trp.

In one embodiment AA₁ is Tyr, AA₂ is Ile, AA₃ is Tyr. In an exemplary embodiment, AA₁ is Tyr, AA₂ is Ile, AA₃ is Tyr, R₁ is H and R₂ is —NH₂ or —NHR₄ where R₄ is C₁-C₁₆ alkyl. R₄ can be C₆ alkyl or C₁₆ alkyl. The compound of the invention can have the formula H-Tyr-Ile-Tyr-NH₂. The compound of the invention can have the formula H-Tyr-Ile-Tyr-NHC₆H₁₃. The compound of the invention can have the formula H-Tyr-Ile-Tyr-NHC₁₆H₃₃. When, AA₁ is Tyr, AA₂ is Ile, AA₃ is Tyr, for example, as in the above-mentioned embodiments, the sum of m, n, p and q can be 0.

In one embodiment AA₁ is Tyr, AA₂ is Leu, AA₃ is Tyr. In an exemplary embodiment, AA₁ is Tyr, AA₂ is Leu, AA₃ is Tyr, R₁ is palmitoyl and R₂ is —NH₂ or —OH. The compound of the invention can have the formula Palm-Tyr-Leu-Tyr-NH₂. The compound of the invention can have the formula Palm-Tyr-Leu-Tyr-OH. When, AA₁ is Tyr, AA₂ is Leu, AA₃ is Tyr, for example, as in the above-mentioned embodiments, the sum of m, n, p and q can be 0.

In one embodiment AA₁ is Tyr, AA₂ is Met, AA₃ is Tyr. In an exemplary embodiment, AA₁ is Tyr, AA₂ is Met, AA₃ is Tyr, R₁ is H and R₂ is —NH₂. For example, the compound of the invention can have the formula H-Tyr-Met-Tyr-NH₂. When, AA₁ is Tyr, AA₂ is Met, AA₃ is Tyr, for example, as in the above-mentioned embodiments, the sum of m, n, p and q can be 0.

In one embodiment, AA₁ is Tyr, AA₂ is selected from the group consisting of Val, Ile, Leu and Met, and AA₃ is Phe. In this embodiment, AA₂ can be Val or Ile.

In one embodiment, AA₁ is Tyr, AA₂ is Val and AA₃ is Phe. In an exemplary embodiment, AA₁ is Tyr, AA₂ is Val or Ile, AA₃ is Phe, R₁ is selected from the group consisting of H and myristoyl, and R₂ is —NH₂ or —NHR₄ where R₄ is C₁-C₁₆ alkyl. R₄ can be C₆ alkyl or C₁₆ alkyl. In an exemplary embodiment, AA₁ is Tyr, AA₂ is Val, AA₃ is Phe, R₁ is myristoyl and R₂ is —NH₂. For example, the compound of the invention can have the formula Myr-Tyr-Val-Phe-NH₂. In an exemplary embodiment, AA₁ is Tyr, AA₂ is Ile, AA₃ is Phe, R₁ is H and R₂ is —NH₂ or —NHR₄ where R₄ is C₁-C₁₆ alkyl. R₄ can be C₆ alkyl or C₁₆ alkyl. The compound of the invention can have the formula H-Tyr-Ile-Phe-NH₂. The compound of the invention can have the formula H-Tyr-Ile-Phe-NHCeH13. The compound of the invention can have the formula H-Tyr-Ile-Phe-NHC16H₃₃. When, AA₁ is Tyr, AA₂ is Val or Ile, AA₃ is Phe, for example, as in the above-mentioned embodiments, the sum of m, n, p and q can be 0.

In one embodiment, AA₁ is Tyr, AA₂ is selected from the group consisting of Val, Ile, Leu and Met, and AA₃ is Trp. In this embodiment, AA₂ can be Val or Leu.

In one embodiment, AA₁ is Tyr, AA₂ is Val and AA₃ is Trp. In an exemplary embodiment, AA₁ is Tyr, AA₂ is Val or Leu, AA₃ is Trp, R₁ is H and R₂ is —NH₂. The compound of the invention can have the formula H-Tyr-Val-Trp-NH₂. The compound of the invention can have the formula H-Tyr-Leu-Trp-NH₂. When, AA₁ is Tyr, AA₂ is Val or Leu, AA₃ is Trp, for example, as in the above-mentioned embodiments, the sum of m, n, p and q can be 0.

In one embodiment, AA₁ is Trp, AA₂ is selected from the group consisting of Val, Ile, Leu or Met, and AA₃ is selected from the group consisting of Tyr, Phe and Trp.

In one embodiment, AA₁ is Trp, AA₂ is selected from the group consisting of Val, Ile, Leu and Met, and AA₃ is selected from the group consisting of Tyr, Phe and Trp.

In one embodiment, AA₁ is Trp, AA₂ is Val, Ile, Leu or Met, and AA₃ is Tyr. In this embodiment, AA₂ can be Val. In an exemplary embodiment, AA₁ is Trp, AA₂ is Val, AA₃ is Tyr, R₁ is acetyl and R₂ is —NH₂. For example, the compound of the invention can have the formula Ac-Trp-Val-Tyr-NH₂. When, AA₁ is Trp, AA₂ is Val, AA₃ is Tyr, for example, as in the above-mentioned embodiments, the sum of m, n, p and q can be 0.

In one embodiment, AA₁ is Trp, AA₂ is selected from the group consisting of Val, Ile, Leu and Met, and AA₃ is Phe.

In one embodiment, AA₁ is Trp, AA₂ is selected from the group consisting of Val, Ile, Leu and Met, and AA₃ is Trp.

Compounds of the invention include those selected from the group of amino acid sequences listed in Table 2, in which their sequence identifier is detailed, their stereoisomers, and/or their cosmetically or pharmaceutically acceptable salts.

TABLE 2

| Sequence | Identifier |
|---|---|
| Tyr-Val-Tyr | PEP-1 |
| Trp-Val-Tyr | PEP-2 |
| Tyr-Ile-Tyr | PEP-3 |
| Tyr-Leu-Tyr | PEP-4 |
| Tyr-Met-Tyr | PEP-5 |
| Tyr-Val-Phe | PEP-6 |
| Tyr-Val-Trp | PEP-7 |
| Tyr-Ile-Phe | PEP-8 |
| Tyr-Leu-Trp | PEP-9 |
| Tyr-Val-Tyr-Phe | SEQ ID NO. 6 |
| Tyr-Val-Tyr-Trp | SEQ ID NO. 7 |
| Phe-Tyr-Val-Tyr-Phe | SEQ ID NO. 8 |
| Phe-Trp-Tyr-Val-Tyr | SEQ ID NO. 9 |
| Tyr-Val-Tyr-Phe-Trp | SEQ ID NO. 10 |

In the amino acid sequences of Table 2 according to formula (1), R₁ and R₂ are H and OH, respectively. Compounds of the invention include each of the sequences of Table 2 with their N- and C-terminals modified by the other R₁ and R₂ groups, respectively, as defined herein for formula (1). For example, compounds of the invention include each of the sequences of Table 2 in which the C-terminal amino acid residue optionally terminates with R₁ as defined above for formula (1), where R₁ is not H. Also, compounds of the invention include each of the sequences of Table 1 in which the N-terminal amino acid residue optionally terminates with R₂ as defined above for formula (1), where R₂ is not OH.

The compounds of this invention can exist as stereoisomers or mixtures of stereoisomers; for example, the amino acids which comprise them can have the configuration L-, D-, or be racemic independently of each other. Therefore, it is possible to obtain isomeric mixtures as well as racemic mixtures or diastereomeric mixtures, or pure diastereomers or enantiomers, depending on the number of asymmetric carbons and on which isomers or isomeric mixtures are present. The preferred structures of the compounds of the invention are pure isomers, i.e., enantiomers or diastereomers.

For example, when it is stated that AA₂ can be Val, it is understood that AA₂ is selected from L-Val, D-Val, and mixtures of both, racemic or non-racemic. The preparation procedures described in this document enable the person skilled in the art to obtain each of the stereoisomers of the compound of the invention by choosing the amino acid with the right configuration.

In the context of this invention, the term “amino acids” includes the amino acids encoded by the genetic code as well as non-encoded amino acids, whether they are natural or not. Examples of non-encoded amino acids are, without restriction, citrulline, ornithine, sarcosine, desmosine, norvaline, 4-aminobutyric acid, 2-aminobutyric acid, 2-aminoisobutyric acid, 6-aminohexanoyc acid, 1-naphthylalanine, 2-naphthylalanine, 2-aminobenzoic acid, 4-aminobenzoic acid, 4-chlorophenylalanine, 2,3-diaminopropionic acid, 2,4-diaminobutyric acid, cycloserine, carnitine, cystine, penicillamine, pyroglutamic acid, thienylalanine, hydroxyproline, allo-isoleucine, allo-threonine, isonipecotic acid, isoserine, phenylglycine, statin, β-alanine, norleucine, N-methyl amino acids, α-amino acids and β-amino acids, among others, as well as their derivatives. A list of non-natural amino acids can be found in the article “Unusual amino acids in peptide synthesis” by D. C. Roberts and F. Vellaccio, in The Peptides, Vol. 5 (1983), Chapter VI, Gross E. and Meienhofer J., Eds., Academic Press, New York, USA or in the commercial catalogues of the companies specialized in the field.

In the context of this invention, when W, X, Y and/or Z are present, i.e., at least one of n, m, p or q is not 0, it is understood that the nature of W, X, Y and/or Z does not hinder the activity of the compound of the invention, and, instead, contributes to it or has no effect on it. In one embodiment, W, X, Y and Z are each independently selected from the group consisting of Phe and Trp.

In one embodiment of the invention each of m, n, p and q is 0, i.e., the compound of formula (I) is a peptide which comprises 3 amino acids, AA₁, AA₂ and AA₃, linked in a chain. In one embodiment the sum of m, n, p and q is 1, i.e., the compound of formula (I) is a peptide which comprises 4 amino acids linked in a chain. In one embodiment the sum of m, n, p and q is 2, i.e., the compound of formula (I) is a peptide which comprises 5 amino acids linked in a chain.

In particular, the compound of the invention can be selected from the group of compounds listed in Table 2a, their stereoisomers, and/or their cosmetically or pharmaceutically acceptable salts.

TABLE 2a
Compound

H-Tyr-Val-Tyr-NH₂
H-Tyr-Val-Tyr-OH
Ac-Tyr-Val-Tyr-NH₂
Palm-Tyr-Val-Tyr-NH₂
Myr-Tyr-Val-Tyr-NH₂
H-Tyr-Val-Tyr-NHC₆H₁₃
H-Tyr-Val-Tyr-NHC₁₆H₃₃
Ac-Trp-Val-Tyr-NH₂
H-Tyr-Ile-Tyr-NH₂
H-Tyr-Ile-Tyr-NHC₆H₁₃
H-Tyr-Ile-Tyr-NHC₁₆H₃₃
Palm-Tyr-Leu-Tyr-NH₂
Palm-Tyr-Leu-Tyr-OH
H-Tyr-Met-Tyr-NH₂
Myr-Tyr-Val-Phe-NH₂
H-Tyr-Val-Trp-NH₂
H-Tyr-Ile-Phe-NH₂
H-Tyr-Ile-Phe-NHC₆H₁₃
H-Tyr-Ile-Phe-NHC₁₆H₃₃
H-Tyr-Leu-Trp-NH₂
H-Tyr-Val-Tyr-Phe-NH₂
H-Tyr-Val-Tyr-Trp-NH₂
H-Phe-Tyr-Val-Tyr-Phe-NH₂
H-Phe-Trp-Tyr-Val-Tyr-NH₂
H-Tyr-Val-Tyr-Phe-Trp-NH₂

The cosmetically or pharmaceutically acceptable salts of the compounds provided by the present invention are also found within the field of this invention. The term “cosmetically or pharmaceutically acceptable salts” means a salt recognized for its use in animals, for example, in mammals, and more specifically in human beings, and includes salts used to form base addition salts, either they are inorganic, for example and not restricted to, lithium, sodium, potassium, calcium, magnesium, manganese, copper, zinc or aluminum among others, or they are organic, for example and not restricted to, ethylamine, diethylamine, ethylenediamine, ethanolamine, diethanolamine, arginine, lysine, histidine or piperazine among others, or acid addition salts, either they are organic, for example and not restricted to, acetate, citrate, lactate, malonate, maleate, tartrate, fumarate, benzoate, aspartate, glutamate, succinate, oleate, trifluoroacetate, oxalate, pamoate or gluconate among others, or inorganic, for example and not restricted to, chloride, sulfate, borate or carbonate, among others. The nature of the salt is not critical, provided that it is cosmetically or pharmaceutically acceptable. The cosmetically or pharmaceutically acceptable salts of the compounds of the invention can be obtained by the conventional methods, well known in the prior art [Berge S. M. et al., “Pharmaceutical Salts”, (1977), J. Pharm. Sci., 66, 1-19].

### Preparation Procedures of the Compounds of the Invention

Synthesis of the compounds of the invention, their stereoisomers, mixtures thereof and/or their cosmetically or pharmaceutically acceptable salts can be carried out according to conventional methods, known in the prior art, such as solid phase peptide synthesis methods [Stewart, J. M., and Young, J. D., “Solid Phase Peptide Synthesis, 2nd edition”, (1984), Pierce Chemical Company, Rockford, Ill.; Bodanzsky, M., and Bodanzsky, A., “The practice of Peptide Synthesis”, (1994), Springer Verlag, Berlin; Lloyd-Williams, P., et al., “Chemical Approaches to the Synthesis of Peptides and Proteins”, (1997), CRC, Boca Raton, Fla., USA], synthesis in solution, enzymatic synthesis [Kullmann, W., “Proteases as catalysts for enzymic syntheses of opioid peptides”, (1980), J. Biol. Chem., 255(17), 8234-8238] or any combination thereof. The compounds can also be obtained by fermentation of a bacterial strain, modified or unmodified by genetic engineering with the objective of producing the desired sequences, or by controlled hydrolysis of proteins with animal or plant origins, preferably plant, which results in free peptide fragments that contain the desired sequence.

For example, a method of obtaining the compounds of formula (I), their stereoisomers and mixtures thereof comprises the stages of:
coupling of an amino acid, with the N-terminal end protected and the C-terminal end free, with an amino acid with the N-terminal end free and the C-terminal end protected or bound to a solid support;
elimination of the protective group of the N-terminal end;
repetition of the coupling sequence and elimination of the protective group of the N-terminal end until the desired peptide sequence is obtained;
elimination of the protective group of the C-terminal end or cleavage of the solid support.

Preferably, the C-terminal end is bound to a solid support and the process is carried out in solid phase and, therefore, comprises the coupling of an amino acid with the N-terminal end protected and the C-terminal end free, with an amino acid with the N-terminal end free and the C-terminal end bound to a polymeric support; elimination of the protective group of the N-terminal end; and repetition of this sequence as many times as is necessary to thus obtain the compound of desired length, finally followed by the cleavage of the synthesized compound from the original polymeric support.

The functional groups of the side chains of the amino acids are maintained conveniently protected with temporary or permanent protective groups throughout synthesis, and can be unprotected simultaneously or orthogonally to the process of cleavage of the peptide from the polymeric support.

Alternatively, solid phase synthesis can be carried out using a convergent strategy coupling a peptide with the polymeric support or with a peptide or an amino acid previously bound to the polymeric support. Convergent synthesis strategies are widely known by persons skilled in the art and are described in Lloyd-Williams P. et al., “Convergent Solid-Phase Peptide Synthesis”, (1993), Tetrahedron, 49(48), 11065-11133.

The process can comprise the additional stages of deprotection of the N-terminal and C-terminal ends and/or cleavage of the peptide from the polymeric support in an indiscriminate order, using standard procedures and conditions known in the prior art, after which the functional groups of these ends can be modified. The optional modification of the N-terminal and C-terminal ends can be carried out with the peptide of formula (I) anchored to the polymeric support or once the peptide has been separated from the polymeric support.

Optionally, R₁ can be introduced by the reaction of the N-terminal end of the compound of the invention with a R₁—X compound through a nucleophilic substitution reaction, in the presence of an adequate base and solvent, wherein the fragments that have the functional groups not involved in the N—C bond formation are suitably protected with temporary or permanent protective groups. R₁ is as defined above and X is a leaving group, for example and not restricted to, the tosyl group, the mesyl group and halogen groups among others.

Optionally and/or additionally, the R₂ radicals can be introduced by the reaction of a compound HR₂ with a complementary fragment which corresponds to the peptide of formula (I) in which R₂ is —OH in the presence of an adequate solvent and a base such as N,N-diisopropylethylamine (DIEA) or trimethylamine, or an additive such as 1-hydroxybenzotriazole (HOBt) or 1-hydroxyazabenzotriazole (HOAt), and a dehydrating agent such as a carbodiimide, a uronium salt, a phosphonium salt or amidinium salt, among others, or by prior formation of an acyl halide with, for example, thionyl chloride, and thereby obtaining a peptide according to the invention of formula (I), wherein the fragments that have the functional groups not involved in the N—C bond formation are suitably protected with temporary or permanent protective groups. Alternatively, other R₂ radicals may be introduced by simultaneous incorporation to the peptide cleavage process from the polymeric carrier. R₂ is —OR₃, —NR₃R₄ or —SR₃, where R₃ and R₄ are as defined above.

A person skilled in the art would easily understand that the deprotection/cleavage steps of the C-terminal and N-terminal ends and their subsequent derivatization can be performed in a different order, according to the processes known in the prior art.

The term “protective group” relates to a group which blocks an organic functional group and which can be removed in controlled conditions. The protective groups, their relative reactivities and the conditions in which they remain inert are known to the person skilled in the art.

Examples of representative protective groups for the amino group are amides, such as amide acetate, amide benzoate, amide pivalate; carbamates such as benzyloxycarbonyl (Cbz or Z), 2-chlorobenzyl (CIZ), para-nitrobenzyloxycarbonyl (pNZ), tert-butyloxycarbonyl (Boc), 2,2,2-trichloroethyloxycarbonyl (Troc), 2-(trimethylsilyl)ethyloxycarbonyl (Teoc), 9-fluorenylmethyloxycarbonyl (Fmoc) or allyloxycarbonyl (Alloc), trityl (Trt), methoxytrityl (Mtt), 2,4-dinitrophenyl (Dnp), N-[1-(4,4-dimethyl-2,6-dioxocyclohex-1-ylidene)ethyl (Dde), 1-(4,4-dimethyl-2,6-dioxo-cyclohexylidene)-3-methylbutyl (ivDde), 1-(1-adamantyl)-1-methylethoxycarbonyl (Adpoc), among others, preferably Boc or Fmoc.

Examples of representative protective groups for the carboxyl group are esters, such as the tert-butyl ester (tBu), allyl ester (All), triphenylmethyl ester (Trt tester), cyclohexyl ester (cHx), benzyl ester (Bzl), ortho-nitrobenzyl ester, para-nitrobenzyl ester, para-methoxybenzyl ester, trimethylsilylethyl ester, 2-phenylisopropyl ester, fluorenylmethyl ester (Fm), 4-(N-[1-(4,4-dimethyl-2,6-dioxocyclohexylidene)-3-methylbutyl]amino) benzyl ester (Dmab), among others; preferred protective groups of the invention are the All, tBu, cHex, Bzl and Trt esters.

The side chains of trifunctional amino acids can be protected during the synthetic process with temporary or permanent protective groups orthogonal to the protective groups of the N-terminal and C-terminal ends.

The hydroxyl group of the tyrosine side chain can be protected with the 2-bromobenzyloxycarbonyl group (2-BrZ), tBu, All, Bzl or 2,6-dichlorobenzyl (2,6-diClZ) among others. In a preferred embodiment, the protective group strategy used is the strategy wherein the amino groups are protected by Boc, the carboxyl groups are protected by Bzl, cHx or All esters and the tyrosine side chain is protected with 2-BrZ or Bzl. In another preferred embodiment, the protective group strategy used is the strategy wherein the amino groups are protected by Fmoc, the carboxyl groups are protected by tBu, All or Trt esters, the tyrosine side chain is protected by tBu.

The amino group of the tryptophan side chain can be protected, for example, by the formyl group (For) or Boc. In one embodiment, when the amino group is protected by Fmoc, and the tryptophan side chain can be: unprotected, i.e., the amino acid is incorporated as Fmoc-Trp-OH; protected by Boc, i.e., the amino acid is incorporated as Fmoc-Trp(Boc)-OH; or protected by For, i.e., the amino acid is incorporated as Fmoc-Trp(For)-OH. In one embodiment, the amino group is protected by Boc, and the tryptophan side chain can be protected by For, i.e., the amino acid is incorporated as Boc-Trp(For)-OH.

Examples of these and other protective groups, their introduction and removal, can be found in the literature [Atherton, B., and Sheppard, R. C., “Solid Phase Peptide Synthesis: A practical approach”, (1989), IRL Oxford University Press]. The term “protective groups” also includes the polymeric supports used in solid phase synthesis.

When synthesis takes place totally or partially in solid phase, the possible solid supports used in the process of the invention involve polystyrene support, polyethylene glycol grafted to polystyrene and similar, for example and not restricted to, p-methylbenzhydrylamine resins (MBHA) [Matsueda, G. R., et al., “A p-methylbenzhydrylamine resin for improved solid-phase synthesis of peptide amides”, (1981), Peptides, 2, 45-50], 2-chlorotrityl resins [Barlos, K., et al., “Darstellung geschützter Peptid-Fragmente unter Einsatz substituierter Triphenylmethyl-Harze”, (1989), Tetrahedron Lett., 30, 3943-3946; Barlos, K., et al., “Veresterung von partiell geschützten Peptid-Fragmenten mit Harzen. Einsatz von 2-Chlorotritylchlorid zur Synthese von Leu1-Gastrin I”, (1989), Tetrahedron Lett., 30, 3947-3951], TentaGel™ resins (Rapp Polymere GmbH), ChemMatrix™ resins (Matrix Innovation, Inc) and similar, which may or may not include a labile linker, such as 5-(4-aminomethyl-3,5-dimethoxyphenoxy) valeric acid (PAL) [Albericio, F., et al., “Preparation and application of the 5-(4-(9-fluorenylmethyloxycarbonyl) aminomethyl-3,5-dimethoxy-phenoxy)valeric acid (PAL) handle for the solid-phase synthesis of C-terminal peptide amides under mild conditions”, (1990), J. Org. Chem., 55, 3730-3743], 2-[4-aminomethyl-(2,4-dimethoxyphenyl)] phenoxyacetic acid (AM) [Rink, H., “Solid-phase synthesis of protected peptide fragments using a trialkoxy-diphenyl-methylester resin”, (1987), Tetrahedron Lett., 28, 3787-3790], [Wang, S. S., “p-Alkoxybenzyl Alcohol Resin and p-Alkoxybenzyloxycarbonylhydrazide Resin for Solid Phase Synthesis of Protected Peptide Fragments”, (1973), J. Am. Chem. Soc., 95, 1328-1333] and similar, which enable simultaneous deprotection and cleavage of the compound from the polymeric support.

### Applications

The present invention is based on the finding that the compound of formula (I) can be used in the treatment of the skin, hair, nails and/or mucous membranes. The treatment can be therapeutic or non-therapeutic. It has been found that the compound of the invention can modulate the level of nocturnin and, in particular, can lower the expression level of nocturnin in subcutaneous pre-adipocyte cells during the night and can reduce lipid accumulation in subcutaneous pre-adipocyte cells. Thus, the present invention also relates the use of the compound of formula (I) in both non-therapeutic and therapeutic treatments of conditions of the skin affected by the presence of nocturnin in the skin, lipid accumulation in the skin and/or adipocyte cell functions. The invention also provides for the use of the compound of formula (I) to modulate nocturnin; the compound of formula (I) can be used to modulate the level (or amount) of nocturnin in the skin. More specifically, the compound of formula (I) can be used to modulate the level of nocturnin in subcutaneous pre-adipocyte cells. The modulation of nocturnin includes the lowering of nocturnin level in the skin and, more specifically, lowering of nocturnin levels in subcutaneous pre-adipocyte cells.

In one aspect, the invention provides the use of the compound of the invention, its stereoisomers and/or its cosmetically acceptable salts, or a cosmetic composition comprising the compound of the invention, its stereoisomers and/or its cosmetically acceptable salts, in the cosmetic, non-therapeutic treatment and/or care of the skin, hair, nails and/or mucous membranes. In particular, the cosmetic, non-therapeutic treatment and/or care is that of the skin. In the context of this invention, skin includes the skin of the whole body including the skin of the face (including skin around the eyes), neckline, neck, décolletage, arms, hands, legs, feet, thighs, hips, buttocks, stomach and torso. The cosmetic, non-therapeutic treatment and/or care can involve the modulation of nocturnin.

The compound of the invention is useful in the cosmetic, non-therapeutic treatment and/or care of the skin, including: the reduction of lipid accumulation in the skin; the treatment and/or prevention of cellulite; the stimulation of lipolysis of the skin; face and neck modelling including reducing the size of or removing a double chin; the stimulation of collagen synthesis; the treatment and/or prevention of the aging of the skin, in particular the treatment of premature aging of the skin associated with the presence of cellulite; the treatment and/or prevention of skin wrinkles; the treatment or prevention of eye bags; maintaining and improving skin firmness; maintaining and improving skin elasticity; promotion of collagen synthesis; the reduction of inflammation; and/or improving BMI value of a subject.

The compound of the invention is useful in the cosmetic, non-therapeutic treatment and/or care of the skin, including: the reduction of lipid accumulation in the skin; the treatment and/or prevention of cellulite; the stimulation of lipolysis of the skin; face and neck modelling including reducing the size of or removing a double chin; the stimulation of collagen synthesis; the treatment and/or prevention of the aging of the skin, in particular the treatment of premature aging of the skin associated with the presence of cellulite; the treatment and/or prevention of skin wrinkles; the treatment or prevention of eye bags; maintaining and improving skin firmness; maintaining and improving skin elasticity; promotion of collagen synthesis; and/or improving BMI value of a subject.

In particular, the compound of the invention is useful in the cosmetic, non-therapeutic treatment and/or care of the skin, including: the reduction of lipid accumulation in the skin; face and neck modelling to improve the aesthetic appearance of the face and including reducing the size of or removing a double chin; the treatment and/or prevention of cellulite; the treatment of premature aging of the skin associated with the presence of cellulite; maintaining and improving skin firmness; maintaining and/or improving skin elasticity.

In one embodiment, there is provided the use of the compound of the invention, its stereoisomers and/or its cosmetically acceptable salts, or a cosmetic composition comprising the compound of the invention, its stereoisomers and/or its cosmetically acceptable salts, for the reduction of lipid accumulation in the skin.

In one embodiment, there is provided the use of the compound of the invention, its stereoisomers and/or its cosmetically acceptable salts, or a cosmetic composition comprising the compound of the invention, its stereoisomers and/or its cosmetically acceptable salts, for the stimulation of lipolysis in the skin.

In one embodiment, there is provided the use of the compound of the invention, its stereoisomers and/or its cosmetically acceptable salts or a cosmetic composition comprising the compound of the invention, its stereoisomers and/or its cosmetically acceptable salts, for the treatment and/or prevention of cellulite. The treatment of cellulite includes the smoothing out of the skin and reduction in the orange peel appearance of the skin due to the presence of cellulite.

In one embodiment, there is provided the use of the compound of the invention, its stereoisomers and/or its cosmetically acceptable salts, or a cosmetic composition comprising the compound of the invention, its stereoisomers and/or its cosmetically acceptable salts, for the cosmetic treatment of inflammation of the skin.

In one embodiment, there is provided the use of the compound of the invention, its stereoisomers and/or its cosmetically acceptable salts or a cosmetic composition comprising the compound of the invention, its stereoisomers and/or its cosmetically acceptable salts, for the treatment and prevention of aging of the skin. This embodiment of the invention provides the cosmetic, non-therapeutic use of the compound of the invention, its stereoisomers and/or its cosmetically acceptable salts or a cosmetic composition comprising the compound of the invention, its stereoisomers and/or its cosmetically acceptable salts, for the alleviation and/or the prevention of symptoms of skin aging. The symptoms of skin aging include the appearance of wrinkles, the appearance of eyebags and the loss of skin biomechanical properties such as firmness and elasticity. The loss of firmness and elasticity can be due to the reduction in collagen production in the skin with age. Skin aging includes, in particular, premature skin aging associated with the presence of cellulite.

In one embodiment, there is provided the use of the compound of the invention, its stereoisomers and/or its cosmetically acceptable salts or a cosmetic composition comprising the compound of the invention, its stereoisomers and/or its cosmetically acceptable salts, for maintaining and/or improving skin firmness.

In one embodiment, there is provided the use of the compound of the invention, its stereoisomers and/or its cosmetically acceptable salts or a cosmetic composition comprising the compound of the invention, its stereoisomers and/or its cosmetically acceptable salts, for maintaining and/or improving skin elasticity.

In one embodiment, there is provided the cosmetic non-therapeutic use of the compound of the invention, its stereoisomers and/or its cosmetically acceptable salts or a cosmetic composition comprising the compound of the invention, its stereoisomers and/or its cosmetically acceptable salts, for the treatment and/or prevention of skin wrinkles.

In one embodiment, there is provided the cosmetic non-therapeutic use of the compound of the invention, its stereoisomers and/or its cosmetically acceptable salts or a cosmetic composition comprising the compound of the invention, its stereoisomers and/or its cosmetically acceptable salts, for the treatment of eye bags. Eye bags can be the result of orbital fat prolapse which occurs with advancing age. In this embodiment, treatment of bags means reducing the volume of eyebags or preventing the appearance of eyebags.

In one embodiment, there is provided the use of the compound of the invention, its stereoisomers and/or its cosmetically acceptable salts or a cosmetic composition comprising the compound of the invention, its stereoisomers and/or its cosmetically acceptable salts, for the promotion or stimulation of collagen synthesis.

In one embodiment, there is provided the use of the compound of the invention, its stereoisomers and/or its cosmetically acceptable salts or a cosmetic composition comprising the compound of the invention, its stereoisomers and/or its cosmetically acceptable salts, for face and neck modelling, for example, reducing the size of or removing a double chin.

In one embodiment, there is provided the use of the compound of the invention, its stereoisomers and/or its cosmetically acceptable salts or a cosmetic composition comprising the compound of the invention, its stereoisomers and/or its cosmetically acceptable salts, for reducing the BMI value of a subject.

In another aspect, the invention provides a cosmetic, non-therapeutic method of treatment and/or care of the skin, hair, nails and/or mucous membranes in a subject comprising administering a cosmetically effective amount of a compound of the invention, its stereoisomers and/or its cosmetically acceptable salts or a cosmetic composition comprising a cosmetically effective amount of the compound of the invention, its stereoisomers and/or its cosmetically acceptable salts, to the subject. In particular the cosmetic, non-therapeutic method of treatment and/or care is that of the skin. The administration can be topical or, for example, transdermal. In this aspect of the invention, the compound of the invention may be present in a cosmetic composition, for example a cosmetic composition as described herein. In one embodiment, the method involves administering the cosmetically effective amount of the compound or administering the composition at night, i.e., during the period from sunset to sunrise.

The cosmetic non-therapeutic method can be for the treatment and/or care of the skin, hair, nails and/or mucous membranes as described above in relation to applications (uses) of the compounds of the invention and cosmetic compositions comprising compounds of the invention.

In another aspect, the invention provides a compound of the invention, its stereoisomers and/or its pharmaceutically acceptable salts or a pharmaceutical composition comprising the compound, its stereoisomers and/or its pharmaceutically acceptable salts for use as a medicament. In particular, the invention provides a compound of the invention, its stereoisomers and/or its pharmaceutically acceptable salts or a pharmaceutical composition comprising the compound, its stereoisomers and/or its pharmaceutically acceptable salts for use in the treatment of a disease or disorder associated with the presence of nocturnin, the accumulation of lipids and/or the function of adipocytes in the skin. In particular, the invention provides a compound of the invention, its stereoisomers and/or its pharmaceutically acceptable salts or a pharmaceutical composition comprising the compound, its stereoisomers and/or its pharmaceutically acceptable salts for use in the treatment of inflammation of the skin. The inflammation can be the result of dysregulation of adipokine production resulting from adipocyte dysfunction which occurs as a result of adipose tissue expansion (which may be due to the over-nutrition or physical inactivity, for example). The use as a medicament or the treatment of a disease or disorder can involve the modulation of nocturnin.

In another aspect, the invention provides for the use of the compound of the invention, its stereoisomers and/or its pharmaceutically acceptable salts for the manufacture of a medicament for the treatment or prevention of a disease or disorder. In particular, the invention provides for the use of the compound of the invention, its stereoisomers and/or its pharmaceutically acceptable salts for the manufacture of a medicament for the treatment of a disease or disorder associated with the presence of nocturnin, the accumulation of lipids and/or the function of adipocytes in the skin, such as inflammation of the skin.

In another aspect, the invention provides a method of treating or preventing a disease or disorder in a subject comprising administering a therapeutically effective amount of a compound of the invention, its stereoisomers and/or its pharmaceutically acceptable salts or a pharmaceutical composition comprising a therapeutically effective amount of compound, its stereoisomers and/or its pharmaceutically acceptable salts, to the subject. In particular, the invention provides for a method of treating inflammation of the skin comprising administering a therapeutically effective amount of a compound of the invention, its stereoisomers and/or its pharmaceutically acceptable salts to the skin. The administration can be topical or, for example, transdermal. In this aspect of the invention, the compound of the invention its stereoisomers and/or its pharmaceutically acceptable salts may be present in a pharmaceutical composition, for example the pharmaceutical compositions described herein.

For the above described methods of the invention, topical or transdermal application can be carried out by iontophoresis, sonophoresis, electroporation, mechanical pressure, osmotic pressure gradient, occlusive cure, microinjections, by needle-free injections by means of pressure, by microelectric patches, face masks or any combination thereof.

For the above described methods of the invention, the frequency of application or administration can vary greatly, depending on the needs of each subject, with a recommendation of an application from once a month to ten times a day, preferably from once a week to four times a day, more preferably from three times a week to twice a day, even more preferably once a day. In one embodiment, the method involves administering the compound of the invention to the subject at night; in particular, this is the case for treatment of those conditions associated with the presence of nocturnin in the skin.

### Cosmetic or Pharmaceutical Compositions of the Invention

The compounds of the invention can be administered for their application by any means that causes contact between the compounds and the site of action in a subject's body, preferably that of a mammal, preferably a human, and in the form of a composition which contains them.

In another aspect, the invention provides a cosmetic or pharmaceutical composition comprising a compound according to formula (I), its stereoisomers and/or its cosmetically or pharmaceutically acceptable salts.

In particular, the invention provides a cosmetic or pharmaceutical composition comprising a compound according to formula (I), its stereoisomers and/or its cosmetically or pharmaceutically acceptable salts, together with at least one cosmetically or pharmaceutically acceptable excipient or adjuvant. These compositions can be prepared by conventional means known to persons skilled in the art [“Harry's Cosmeticology”, Seventh edition, (1982), Wilkinson, J. B., Moore, R. J., eds., Longman House, Essex, GB].

The compounds of this invention have variable solubility in water, according to the nature of their amino acid sequence or any possible modifications in the N-terminal and/or C-terminal ends. Therefore, the compounds of this invention can be incorporated into the compositions by aqueous solution, and those which are not soluble in water can be solubilized in cosmetically or pharmaceutically acceptable conventional solvents such as and not restricted to, ethanol, propanol, isopropanol, propylene glycol, glycerin, butylene glycol or polyethylene glycol or any combination thereof.

The cosmetically or pharmaceutically (therapeutically) effective amount of the compounds of the invention which should be administered, as well as their dosage, will depend on numerous factors, including age, state of the patient, the nature or severity of the condition, disorder or disease to be treated and/or cared for, the route and frequency of administration and of the particular nature of the compounds to be used.

The terms “cosmetically effective amount” and “pharmaceutically effective amount” are understood to mean a non-toxic but sufficient amount of the compound or compounds of the invention to provide the desired effect. The terms “pharmaceutically effective” and “therapeutically effective” are used interchangeably herein. The compounds of the invention are used in the cosmetic or pharmaceutical compositions of this invention at cosmetically or pharmaceutically effective concentrations to achieve the desired effect; for example in amounts with respect to the total weight of the composition of: from 0.00000001% (in weight) to 20% (in weight); from 0.000001% (in weight) to 15% (in weight), from 0.00001% (in weight) to 10% (in weight); or from 0.0001% (in weight) to 5% (in weight).

The compounds of formula (I), their stereoisomers, mixtures thereof and/or their cosmetic or pharmaceutically acceptable salts, can also be incorporated into cosmetic or pharmaceutical delivery systems and/or sustained release systems.

The term “delivery system” relates to a diluent, adjuvant, excipient or carrier with which the compound of the invention is administered. These cosmetic or pharmaceutical carriers can be liquids, such as water, oils or surfactants, including those of petroleum, animal, plant or synthetic origin, for example and not restricted to, peanut oil, soybean oil, mineral oil, sesame oil, castor oil, polysorbates, sorbitan esters, ether sulfates, sulfates, betaines, glycosides, maltosides, fatty alcohols, nonoxynols, poloxamers, polyoxyethylenes, polyethylene glycols, dextrose, glycerol, digitonin and similar. A person skilled in the art knows the diluents, adjuvants or excipients which can be used in the different delivery systems in which the compound of the invention can be administered.

The term “sustained release” is used in a conventional sense relating to a delivery system of a compound which provides the gradual release of this compound during a period of time and preferably, although not necessarily, with relatively constant compound release levels over a period of time.

Examples of delivery or sustained release systems include, without restriction, liposomes, mixed liposomes, oleosomes, niosomes, ethosomes, milliparticles, microparticles, nanoparticles and solid lipid nanoparticles, nanostructured lipid carriers, sponges, cyclodextrins, vesicles, micelles, mixed micelles of surfactants, surfactant-phospholipid mixed micelles, millispheres, microspheres and nanospheres, lipospheres, millicapsules, microcapsules and nanocapsules, as well as in microemulsions and nanoemulsions, which can be added to achieve a greater penetration of the active principle and/or improve its pharmacokinetic and pharmacodynamic properties. Preferred delivery or sustained release systems are liposomes, surfactant-phospholipid mixed micelles, microemulsions, more preferably water-in-oil microemulsions with an internal structure of reverse micelle and nanocapsules containing microemulsions.

In one embodiment, the invention provides a cosmetic or pharmaceutical composition comprising a compound of formula (I) and a cosmetically or pharmaceutically acceptable carrier selected from the group consisting of creams, emulsions, gels, liposomes, nanoparticles and ointments.

The sustained release systems can be prepared by methods known in the prior art, and the compositions which contain them can be administered, for example, by topical or transdermal administration, including adhesive patches, non-adhesive patches, occlusive patches and microelectric patches, or by systemic administration, for example and not restricted to, oral or parenteral route, including nasal, rectal or subcutaneous implantation or injection, or direct implantation or injection into a specific body part, and preferably should release a relatively constant quantity of the compounds of the invention. The amount of compound contained in the sustained release system will depend, for example, on where the composition is to be administered, the kinetics and duration of the release of the compound of the invention, as well as the nature of the condition, disorder and/or disease to be treated and/or cared for.

The compounds of this invention can also be adsorbed on solid organic polymers or solid mineral supports such as and not restricted to, talc, bentonite, silica, starch or maltodextrin among others.

The compositions which contain the compounds of formula (I), their stereoisomers, mixtures thereof and/or their cosmetically or pharmaceutically acceptable salts can also be incorporated into fabrics, non-woven fabrics and medical devices which are in direct contact with the skin, thus releasing the compounds of the invention whether by biodegradation of the binding system to the fabric, non-woven fabric or medical device, or by friction between them and the body, due to bodily moisture, the skin's pH or body temperature. Furthermore, the compounds of the invention can be incorporated into the fabrics and non-woven fabrics used to make garments that are in direct contact with the body.

Examples of fabrics, non-woven fabrics, garments, medical devices and means for immobilizing the compounds to them, among which are the delivery systems and/or the sustained release systems described above, can be found in literature and are known in the prior art [Schaab, C. K. (1986) HAPPI, May 1986; Nelson, G., “Application of microencapsulation in textiles”, (2002), Int. J. Pharm., 242(1-2), 55-62; “Biofunctional Textiles and the Skin” (2006) Curr. Probl. Dermatol. v. 33, Hipler, U. C. and Elsner, P., eds., S. Karger AG, Basel, Switzerland; Malcolm, R. K., et al., “Controlled release of a model antibacterial drug from a novel self-lubricating silicone biomaterial”, (2004), J. Cont. Release, 97(2), 313-320]. The preferred fabrics, non-woven fabrics, garments and medical devices are bandages, gauzes, t-shirts, socks, tights, underwear, girdles, gloves, diapers, sanitary napkins, dressings, bedspreads, wipes, adhesive patches, non-adhesive patches, occlusive patches, microelectric patches and/or face masks.

The cosmetic or pharmaceutical compositions which contain the compounds of the invention, their stereoisomers, mixtures thereof and/or their cosmetically or pharmaceutically acceptable salts, can be used in different types of compositions for topical or transdermal application which optionally include cosmetically or pharmaceutically acceptable excipients necessary for formulating the desired administration form.

The compositions for topical or transdermal application can be produced in any solid, liquid or semisolid formulation, such as and not restricted to, creams, multiple emulsions such as and not restricted to, oil and/or silicone in water emulsions, water-in-oil and/or silicone emulsions, water/oil/water or water/silicone/water type emulsions and oil/water/oil or silicone/water/silicone type emulsions, anhydrous compositions, aqueous dispersions, oils, milks, balsams, foams, lotions, gels, cream gels, hydroalcoholic solutions, hydroglycolic solutions, hydrogels, liniments, sera, soaps, shampoos, conditioners, serums, polysaccharide films, ointments, mousses, pomades, powders, bars, pencils and sprays or aerosols (sprays), including leave-on and rinse-off formulations. These topical or transdermal application formulations can be incorporated using techniques known by the person skilled in the art into different types of solid accessories for example and not restricted to, bandages, gauzes, t-shirts, socks, tights, underwear, girdles, gloves, diapers, sanitary napkins, dressings, bedspreads, wipes, adhesive patches, non-adhesive patches, occlusive patches, microelectric patches or face masks, or they can be incorporated into different make-up products such as make-up foundation, such as fluid foundations and compact foundations, make-up removal lotions, make-up removal milks, under-eye concealers, eye shadows, lipsticks, lip protectors, lip gloss and powders among others.

The cosmetic or pharmaceutical compositions of the invention may include agents which increase the percutaneous absorption of the compounds of the invention, for example and not restricted to, dimethylsulfoxide, dimethylacetamide, dimethylformamide, surfactants, azone (1-dodecylazacycloheptane-2-one), alcohol, urea, ethoxydiglycol, acetone, propylene glycol or polyethylene glycol, among others. Furthermore, the cosmetic or pharmaceutical compositions of this invention can be applied to local areas to be treated by means of iontophoresis, sonophoresis, electroporation, microelectric patches, mechanical pressure, osmotic pressure gradient, occlusive cure, microinjections or needle-free injections by means of pressure, such as injections by oxygen pressure, or any combination thereof, to achieve a greater penetration of the peptide of the invention. The application area will be determined by the nature of the condition, disorder and/or disease to be treated and/or cared for.

Furthermore, the cosmetic compositions containing the compounds of formula (I), their stereoisomers, mixtures thereof and/or their cosmetically or pharmaceutically acceptable salts can be used in different types of formulations for oral administration, preferably in the form of oral cosmetics or drugs, such as and not restricted to, capsules, including gelatin capsules, soft capsules, hard capsules, tablets, including sugar coated tablets, tablets, pills, powders, granules, chewing gum, solutions, suspensions, emulsions, syrups, elixirs, polysaccharide films, jellies or gelatins, and any other form known by the person skilled in the art. In a particular embodiment, the compounds of the invention can be incorporated into any form of functional food or fortified food, such as and not restricted to, dietary bars or compact or non-compact powders. These powders can be dissolved in water, soda, dairy products, soy derivatives or can be incorporated into dietary bars. The compounds of this invention can be formulated with common excipients and adjuvants for oral compositions or food supplements, for example and not restricted to, fat components, aqueous components, humectants, preservatives, texturizing agents, flavors, aromas, antioxidants and colorants common in the food industry.

Cosmetic or pharmaceutical compositions containing the compounds of formula (I), their stereoisomers, mixtures thereof and/or their cosmetically or pharmaceutically acceptable salts can also be administered, as well as by topical or transdermal route, by any other appropriate route, such as oral or parenteral route, for which they will include the pharmaceutically acceptable excipients necessary for the formulation of the desired administration form. In the context of this invention, the term “parenteral” includes nasal, auricular, ophthalmic, rectal, urethral, vaginal, subcutaneous, intradermal route, intravascular injections, such as intravenous, intramuscular, intraocular, intravitreous, intracorneal, intraspinal, intramedullary, intracranial, intracervical, intracerebral, intrameningeal, intraarticular, intrahepatic, intrathoracic, intratracheal, intrathecal and intraperitoneal, and any another similar injection or infusion technique. A person skilled in the art knows the different means by which the cosmetic or pharmaceutical compositions which contain the compounds of the invention can be administered.

Among the cosmetically or pharmaceutically acceptable adjuvants contained in the cosmetic or pharmaceutical compositions described in this invention are additional ingredients commonly used in cosmetic or pharmaceutical compositions, for example and not restricted to other nocturnin modulating agents, agents that boost mitochondrial metabolism, agents that enhance adiponectin release, agents that boost intercellular communication, agents that increase connexins in skin cells, agents that promote self-renewal of the skin, agents that prevent hypertrophic scarring of the skin, DNA protecting agents, DNA repair agents, stem cell protecting agents, agents inhibiting neuronal exocytosis, anticholinergic agents, agents inhibiting muscular contraction, antiaging agents, anti-wrinkle agents, antiperspirant agents, anti-inflammatory and/or analgesic agents, anti-itching agents, calming agents, anesthetic agents, inhibitors of acetylcholine-receptor aggregation, inhibitors of acetylcholinesterase, skin relaxant agents, melanin synthesis stimulating or inhibiting agents, whitening or depigmenting agents, propigmenting agents, self-tanning agents, NO-synthase inhibiting agents, 5α-reductase inhibiting agents, lysyl- and/or prolyl hydroxylase inhibiting agents, antioxidants, free radical scavengers and/or agents against atmospheric pollution, reactive carbonyl species scavengers, anti-glycation agents, detoxifying agents, antihistamine agents, antiviral agents, antiparasitic agents, emulsifiers, emollients, organic solvents, liquid propellants, skin conditioners, humectants, substances which retain moisture, alpha hydroxy acids, beta hydroxy acids, moisturizers, hydrolytic epidermal enzymes, vitamins, amino acids, proteins, pigments or colorants, dyes, biopolymers, gelling polymers, thickeners, surfactants, softening agents, emulsifiers, binding agents, preservatives, agents able to reduce or treat the bags under the eyes, exfoliating agents, keratolytic agents, desquamating agents, antimicrobial agents, antifungal agents, fungistatic agents, bactericidal agents, bacteriostatic agents, agents stimulating the synthesis of dermal or epidermal macromolecules and/or capable of inhibiting or preventing their degradation, collagen synthesis-stimulation agents, elastin synthesis-stimulation agents, decorin synthesis-stimulation agents, laminin synthesis-stimulation agents, defensin synthesis-stimulating agents, chaperone synthesis-stimulating agents, cAMP synthesis-stimulating agents, AQP-3 modulating agents, aquaporin synthesis-stimulating agents, proteins of the aquaporin family, hyaluronic acid synthesis-stimulating agents, glycosaminoglycan synthesis-stimulating agents, fibronectin synthesis-stimulating agents, sirtuin synthesis-stimulating agents, sirtuin-activating agents, heat shock proteins, heat shock protein synthesis-stimulating agents, agents stimulating the synthesis of lipids and components of the stratum corneum, ceramides, fatty acids, agents that inhibit collagen degradation, agents that inhibit matrix metalloproteinase, agents that inhibit elastin degradation, agents that inhibit serine proteases such as kallikreins, elastase or cathepsin, agents stimulating fibroblast proliferation, agents stimulating keratinocyte proliferation, agents stimulating adipocyte proliferation, agents stimulating melanocyte proliferation, agents stimulating keratinocyte differentiation, agents stimulating or delaying adipocyte differentiation, antihyperkeratosis agents, comedolytic agents, anti-psoriatic agents, stabilizers, agents for the treatment and/or care of sensitive skin, firming agents, anti-stretch mark agents, binding agents, agents regulating sebum production, lipolytic agents or agents stimulating lipolysis, adipogenic agents, agents modulating PGC-1α expression, agents modulating the activity of PPARγ, agents which increase or reduce the triglyceride content of adipocytes, anti-cellulite agents, agents which inhibit PAR-2 activity, agents stimulating healing, coadjuvant healing agents, agents stimulating reepithelialization, coadjuvant reepithelialization agents, cytokine growth factors, agents acting on capillary circulation and/or microcirculation, agents stimulating angiogenesis, agents that inhibit vascular permeability, venotonic agents, agents acting on cell metabolism, agents to improve dermal-epidermal junction, agents inducing hair growth, hair growth inhibiting or retardant agents, agents delaying hair loss, preservatives, perfumes, cosmetic and/or absorbent and/or body odor-masking deodorants, chelating agents, plant extracts, essential oils, marine extracts, agents obtained from a biotechnological process, mineral salts, cell extracts, sunscreens and organic or mineral photoprotective agents active against ultraviolet A and/or B rays and/or infrared A rays, or mixtures thereof, provided they are physically and chemically compatible with the rest of components of the composition and in particular with the compounds of the invention. Furthermore, the nature of these additional ingredients should not unacceptably alter the benefits of the compounds of this invention. The nature of these additional ingredients can be synthetic or natural, such as plant extracts, or come from a biotechnological process or from a combination of a synthetic procedure and biotechnological process. Additional examples can be found in CTFA International Cosmetic Ingredient Dictionary & Handbook, 12th Edition (2008). In the context of this invention, biotechnological process is understood to be any process that produces the active ingredient, or part of it, in an organism, or in part of it.

In one embodiment, the invention provides a cosmetic or pharmaceutical composition comprising a compound of formula (I) and a pharmaceutically or cosmetically effective amount of an adjuvant selected from the group consisting of:
(i) an agent that reduces the triglyceride content of adipocytes, agent that delays adipocyte differentiation, anti-cellulite agent, lipolytic agent, venotonic agent, agent inhibiting PGC-1α expression or agent inhibiting the activity of PPARγ;
(ii) a firming and/or redensifying and/or restructuring agent;
(iii) an agent that stimulates the synthesis of dermal or epidermal macromolecules;
(iv) an anti-wrinkle and/or antiaging agent;
(v) an anti-inflammatory agent and/or analgesic;
(vi) an agent capable of filtering UV and IRA rays;
(vii) a whitening or depigmenting agent or melanin synthesis inhibiting agent;
(viii) a DNA protecting agent, DNA repair agent, and/or stem cell protecting agent; and
(ix) a reactive carbonyl species scavenger, free radical scavengers and/or anti-glycation agent, detoxifying agent, antioxidant and/or anti-pollution agent.

The cosmetic or pharmaceutical composition can further comprise an agent that reduces the triglyceride content of adipocytes, agent that delays adipocyte differentiation, anti-cellulite agent, lipolytic agent, venotonic agent, agent inhibiting PGC-1α expression or agent inhibiting the activity of PPARγ selected, for example and not restricted to, from the group consisting of extracts or hydrolyzed extracts of Alchemilla vulgaris, Angelica sinensis, Armeniacea sp., Arnica montana L, Atractylodis platycodon, bamboo, Betula alba, Bupleurum chinensis, Calendula officinalis, cangzhu, Cecropia obtusifolia, Celosia cristata, Centella asiatica, Chenopodium quinoa, Chrysanthellum indicum, Cimifuga racemosa, Citrus aurantium amara, Cnicus benedictus, Coffea arabica, Cola nipida, Coleus barbatus, Coleus blumei, Coleus esquirolii, Coleus forskohlii, Coleus scutellarioides, Coleus sp., Coleus xanthanthus, Commiphora myrrha, Crithmum maritimum, Cuminum cyminum, Dioscorea collettii, Dioscorea villosa, Eugenia caryophyllus, Filipendula ulmaria L, Foeniculum vulgare, Fucus vesiculosus, Gelidium Cartilagineum, Ginkgo biloba, Ginkgo biloba, Glycine max, Glycyrrhiza glabra, Hedera helix (ivy extract), Hibiscus sabdariffa, Hordeum vulgare, Humulus lupulus, Hypericum perforatum, Ilex paraguariensis, Kigelia africana, Laminaria digitata, Lupinus perennis, Nelumbium speciosum, Orthosiphon stamineus benth, Panax ginseng, Paullinia cupana, Peumus boldus, Phyllacantha fibrosa, Piper methysticum, Piper nigrum, Prunella vulgaris, Prunus amygdalus dulcis, Rosmarinus officinalis, Rubus idaeus, Ruscus aculeatus (extract of Butcher's broom), Salvia officinalis L, Sambucus nigra, Serenoa repens, Smilax aristolochiaefolia, Spirulina platensis algae, Taraxacum erythrospermum, Taraxacum officinale, green tea, Ulmus rubra, Uncaria tomentosa, Verbena officinalis, Vitex agnus-castus, Dysmorphococcus globosus, among others, alverin, alverin citrate, dihydromyricetin, coenzyme A, lipase, cerulenin, rutin, glaucine, esculin, visnadine, caffeine, theophylline, theobromine, aminophylline, xanthine, carnitine, forskolin, escin, ruscogenin, hederin, triethanolamine iodide, AMPc synthesis inducing agents, Lanachrys® [INCI: Chrysanthellum indicum Extract] marketed by Atrium/Unipex; Slim-Excess™ [INCI: Hydrolyzed Carrageenan, Xanthan Gum], Sveltine™ [INCI: Carnitine, Lecithin, Caffeine, Carbomer, Salicylic Acid, Atelocollagen, Centella asiatica Extract, Esculin, Sodium Chondroitin Sulfate], Peru Liana [INCI: Uncaria tomentosa Extract], Flavenger™ [INCI: Caprylic/Capric Triglyceride, Silica Dimethyl Silylate, Glyceryl Oleate, Quercetin Caprylate] or Slimfit® LS 9509 [INCI: Cecropia obtusifolia Bark Extract] marketed by BASF; Scopariane® [INCI: Sphacelaria Scoparia], Phyco™ R₇₅ [INCI: Laminaria digitata], Pheoslim™ [INCI: Phyllacantha fibrosa Extract], Buckwheat Wax [INCI: Polygonum Fagopyrum], Areaumat™ Samphira [INCI: Crithmum maritimum Extract] or Actiporine™ 8G [Jania Rubens Extract] marketed by Codif; Slimming Factor Karkade™ [INCI: Hibiscus sabdariffa] marketed by Cosmetochem; Liposuctionine™ [Acetyl Cyclohexapeptide-34] marketed by Infinitec Activos; Xantalgosil C® [INCI: Acefylline Methylsilanol Mannuronate], Theophyllisilane C® [INCI: Methylsilanol Carboxymethyl Theophylline Alginate], Glutrapeptide® [INCI: Pyroglutamylamidoethyl Indole] or Cafeisilane™ C [INCI: Siloxanetriol Alginate, Caffeine] marketed by Exsymol; Timiline® [INCI: Polyglucuronic Acid] marketed by Greentech; Visnadine [INCI: Visnadine] or Ginkgo biloba Dimeric Flavonoids Phytosome [INCI: Phospholipids, Ginkgo biloba Leaf Extract] marketed by Indena; Silusyne® [INCI: Soybean (Glycine soja) Oil, Sorbitan Sesquioleate, Isohexadecane, Sodium Hyaluronate, Lauryldimonium Hydroxypropyl Hydrolyzed Soy Protein, Acetyl Hexapeptide-39], Liporeductyl® [INCI: Lecithin, Caffeine, Butcher's broom (Ruscus aculeatus) Root Extract, Maltodextrin, Silica, Tea-Hydroiodide, Ivy (Hedera helix) Extract, Carnitine, Escin, Tripeptide-1, Xanthan Gum, Chondrus Crispus (Carrageenan) Extract], Actigym™ [INCI: Bacillus/Soybean Ferment Extract] or Nocturshape™ [INCI: Saccharide Isomerate] marketed by Lipotec/Lubrizol; Iso-Slim Complex™ [INCI: Soy Isoflavones, Caffeine, Carnitine, Spirulina platensis Extract], Happybelle-PE™ [INCI: Lecithin, Vitex agnus castus Extract, Ascorbyl Tetraisopalmitate, Tocopherol, Caprylic/Capric Triglyceride, Cyclodextrin] or AmaraShape™ [INCI: Lecithin, Caffeine, Citrus aurantium amara Extract] marketed by Mibelle Biochemistry; Regu®-Slim [INCI: Caffeine, Paullinia cupana Seed Extract, Carnitine, Microcrystalline Cellulose, Cysteic Acid, Pantheine Sulfonate] or Regu®-Shape [INCI: Isomerized Linoleic Acid, Lecithin] marketed by Pentapharm/DSM; Provislim™ [INCI: Fisetin, Raspberry Ketone], Myriceline™ [INCI: Dihydromyricetin], Drenalip™ [INCI: Ruscus aculeatus Root Extract, Citrus medica limonum Peel Extract, Solidago Virgaurea Extract, Astragalus Membranaceus Root Extract] or Lipout™ [INCI: Caprylic/Capric Glycerides, Plankton Extract] marketed by Provital; Actisculpt™ [INCI: Commiphora myrrha Extract, Coleus forskohlii Root Extract] marketed by Givaudan; Perfeline® [INCI: Carnitine, Caffeine, Ruscus aculeatus Extract] or CellActive® Shape [INCI: Chlorella vulgaris/Lupinus albus Protein Ferment, Coleus forskohlii, Caffeine] marketed by Rahn; ProContour™ [INCI: Lecithin, Caffeine, Carnitine, Centella asiatica Leaf Extract, Coleus forskohlii Root Extract] marketed by Rovi Cosmetics; Unislim™ [INCI: Ilex paraguariensis (Leaf) Extract, Coffea arabica (Coffee) Seed Extract (Bean), PEG-60 Almond Glycerides, Cetyl Hydroxyethylcellulose], Redulite™ [INCI: Ethoxydiglycol, Sambucus nigra, Sodium Polyacrylate], Pleurimincyl™ [INCI: Caffeine, Bupleurum chinensis Extract], Phytotal™ SL [INCI: Verbena officinalis Extract, Sambucus nigra Flower Extract, Eugenia caryophyllus (Clove) Flower Extract, Lecithin], Phytosonic™ [INCI: Euglena Gracilis Extract, Caffeine, Glaucium Flavum Leaf Extract], Ovaliss™ [INCI: Coco-Glucoside, Glaucine], Lipocare™ [INCI: Caffeine, Coenzyme A, Bupleurum chinensis Extract], Cyclolipase™ [INCI: Glyceryl Polymethacrylate, Caffeine, Lipase, Adenosine Phosphate], Coaxel™ [INCI: Caffeine, Coenzyme A, Carnitine], Bodyfit™ [INCI: Coco-Glucoside, Glaucine], Vexel™ [INCI: Lecithin, Caffeine, Palmitoyl Carnitine] or Intenslim™ [INCI: Globularia Cordifolia Callus Culture Extract, Zingiber Zerumbet Extract, Caffeine] marketed by Sederma/Croda; Voluform™ [INCI: Palmitoyl Isoleucine], Adipoless™ [INCI: Chenopodium quinoa Seed Extract] marketed by Seppic; Slimactive® [INCI: Peumus boldus Leaf Extract], Remoduline® [INCI: Citrus aurantium amara Flower Extract], Pro-Sveltyl™ [INCI: Nelumbium speciosum Extract], Biosculptine® [INCI: Hydrolyzed Celosia cristata Flower/Seed Extract, Hydrolyzed Prunella vulgaris Extract], Affiness® [INCI: Hydrolyzed Coriandrum Sativum Fruit Extract, Citrus aurantium dulcis (Orange) Fruit Extract] or Stemsvelt™ [INCI: Silybum marianum Extract] marketed by Silab; Delipidol™ [INCI: Tyrosyl Punicate], Guaraslim® [INCI: Caffeine, Paullinia cupana Seed Extract, Ptychopetalum Olacoides Bark Extract], Caobromine® [INCI: Theobroma Cocoa Shell Extract] or Slimastevia® [INCI: Ilex paraguariensis Leaf Extract, Stevia Rebaudiana Extract] marketed by Solabia; Abdoliance™ [INCI: Sucrose Palmitate, Glyceryl Linolenate, Paullinia cupana Seed Extract, Prunus amygdalus dulcis (Sweet Almond) Oil, Lecithin, Citrus aurantium amara (Bitter Orange) Peel Extract, Tocopherol], Betaphroline™ [INCI: Tephrosia Purpurea Seed Extract] or PRO-DG [INCI: Plankton Extract] marketed by Soliance; UCPeptide™ V [INCI: Pentapeptide] or ATPeptide™ IS [INCI: Tripeptide-3] marketed by Vincience/ISP/Ashland among others, or mixtures thereof.

The cosmetic or pharmaceutical composition can further comprise a firming and/or redensifying and/or restructuring agent selected, for example and not restricted to, from the group consisting of extracts of Malpighia punicifolia, Cynara scolymus, Gossypium herbaceum, Aloe barbadensis, Panicum miliaceum, Morus nigra, Sesamum indicum, Glycine soja, Triticum vulgare, Pronalen® Refirming HSC [INCI: Triticum vulgare, Silybum marianum, Glycine Soy, Equisetum arvense, Alchemilla vulgaris, Medicago sativa, Raphanus sativus] or Polyplant® Refirming [INCI: Coneflower, Asiatic centella, Fucus, Fenugreek] marketed by Provital; Lanablue® [INCI: Sorbitol, Algae Extract] marketed by Atrium Biotechnologies/Unipex Innovations; Pepha®-Nutrix [INCI: Natural Nutrition Factor] marketed by Pentapharm/DSM; plant extracts containing isoflavones; Biopeptide EL™ [INCI: Palmitoyl Oligopeptide], Biopeptide CL™ [INCI: Palmitoyl Oligopeptide], Vexel® [INCI: Lecithin, Caffeine, Palmitoyl Carnitine], Matrixyl® [INCI: Palmitoyl Pentapeptide-3], Matrixyl® 3000 [INCI: Palmitoyl Tetrapeptide-3, Palmitoyl Oligopeptide], Bio-Bustyl™ [INCI: Glyceryl Polymethacrylate, Rahnella Soy Protein Ferment, Palmitoyl Oligopeptide] or Majestem™ [INCI: Leontopodium alpinum callus Culture Extract] marketed by Sederma/Croda; Dermosaccharides® HC [INCI: Glycosaminoglycans, Glycogen], Aglycal® [INCI: Mannitol, Cyclodextrin, Glycogen, Arctostaphylos Uva Ursi Leaf Extract], Cytokinol® LS [INCI: Hydrolyzed Casein, Hydrolyzed Yeast Protein, Lysine HCl], Firmiderm® LS9120 [INCI: Terminalia Catappa Leaf Extract, Sambucus negra Flower Extract, PVP, Tannic Acid], Ursolisome® [INCI: Lecithin, Ursolic Acid, Atelocollagen, Xanthan Gum, Sodium Chondroitin Sulfate] or Collalift® [INCI: Hydrolyzed Malt Extract] marketed by BASF; Liftline® [INCI: Hydrolyzed Wheat Protein], Raffermine® [INCI: Hydrolyzed Soy Flour], Ridulisse C® [Hydrolyzed Soy Protein] or Volunage® [INCI: Paeonia Albiflora Root Extract] marketed by Silab; Serilesine® [INCI: Hexapeptide-10], Decorinyl® [INCI: Tripeptide-10 Citrulline], Trylagen® [INCI: Pseudoalteromonas Ferment Extract, Hydrolyzed Wheat Protein, Hydrolyzed Soy Protein, Tripeptide-10 Citrulline, Tripeptide-1], Silusyne® [INCI: Soybean (Glycine soja) Oil, Sorbitan Sesquioleate, Isohexadecane, Sodium Hyaluronate, Lauryldimonium Hydroxypropyl Hydrolyzed Soy Protein, Acetyl Hexapeptide-39], Adifyline® [INCI: Acetyl Hexapeptide-38] or Uplevity™ [INCI: Acetyl Tetrapeptide-2] marketed by Lipotec/Lubrizol; Syn®-Coll [INCI: Palmitoyl Tripeptide-5] marketed by Pentapharm/DSM; Hydriame® [INCI: Glycosaminoglycans, Sclerotium Gum] marketed by Atrium Biotechnologies/Unipex Innovations; IP2000™ [INCI: Dextran, Trifluoroacetyl Tripeptide-2] marketed by Institut Européen de Biologie Cellulaire/Unipex Innovations; Plantasens® Olive Active HP [Olea Europaea (Olive) Oil Unsaponifiables] marketed by Clariant, among others.

The cosmetic or pharmaceutical composition can further comprise an agent that stimulates the synthesis of dermal or epidermal macromolecules selected, for example and not restricted to, from the group consisting of collagen synthesis-stimulating agents, elastin synthesis-stimulating agents, decorin synthesis-stimulating agents, laminin synthesis-stimulating agents, chaperone synthesis-stimulating agents, sirtuin synthesis-stimulating agents, sirtuin activating agents, aquaporin synthesis-modulating agents, fibronectin synthesis-stimulating agent, agents that inhibit collagen degradation, agents that inhibit elastin degradation, agents that inhibit serine proteases such as kallikreins, leukocyte elastase or cathepsin G, agents stimulating fibroblast proliferation, and DNA repairing agents and/or DNA protecting agents, such as and not restricted to extracts of Centella asiatica, Saccharomyces cerevisiae, Solanum tuberosum, Rosmarinus officinalis, Vaccinium angustifolium, extract of the algae Macrocystis pyrifera, Padina pavonica, extract of soy, malt, flax, sage, red clover, kakkon, white lupin plants, hazelnut extract, maize extract, yeast extract, beech shoot extracts, leguminous seed extract, plant hormone extract such as gibberellins, auxins or cytokinins, among others, or extract of zooplankton Salina, the fermentation product of milk with Lactobacillus Bulgaricus, asiaticosides and their derivatives, vitamin C and its derivatives, cinnamic acid and its derivatives, Matrixyl® [INCI: Palmitoyl Pentapeptide-3], Matrixyl® 3000 [INCI: Palmitoyl Tetrapeptide-3, Palmitoyl Oligopeptide], Biopeptide CL™ [INCI: Glyceryl Polymethacrylate, Palmitoyl Oligopeptide] or Senestem™ [INCI: Plantago Lanceolata Leaf Extract] marketed by Sederma/Croda; Antarcticine® [INCI: Pseudoalteromonas Ferment Extract], Decorinyl® [INCI: Tripeptide-10 Citrulline], Serilesine® [INCI: Hexapeptide-10], Lipeptide™ [INCI: Hydrolyzed Vegetable Protein], Aldenine® [INCI: Hydrolyzed Wheat Protein, Hydrolyzed Soy Protein, Tripeptide-1], Relistase® [INCI: Acetylarginyltriptophyl Diphenylglycine], Thermostressine® [INCI: Acetyl Tetrapeptide-22], Peptide AC29 [INCI: Acetyl Tripeptide-30 Citrulline], Diffuporine® [INCI: Acetyl Hexapeptide-37], Silusyne® [INCI: Soybean (Glycine soja) Oil, Sorbitan Sesquioleate, Isohexadecane, Sodium Hyaluronate, Lauryldimonium Hydroxypropyl Hydrolyzed Soy Protein, Acetyl Hexapeptide-39], Adifyline® [INCI: Acetyl Hexapeptide-38] or Juveleven™ [INCI: Acetyl Hexapeptide-51 Amide] marketed by Lipotec/Lubrizol; Drieline® PF [INCI: Yeast Betaglucan] or Repulpami™ ER [INCI: Adansonia Digitata Pulp Extract, Hibiscus sabdariffa Flower Extract] marketed by Alban Muller; Phytovityl C® [INCI: Zea mays Extract] marketed by Solabia, Collalift® [INCI: Hydrolyzed Malt Extract], Eperuline™ [INCI: Eperua Falcata Bark Extract] or Shadownyl™ [INCI: Algae Extract] marketed by BASF; Phytocohesine PSP™ [INCI: Sodium Beta-Sitosterol Sulfate] or GP4G™ SP [INCI: Artemia Extract] marketed by Vincience/ISP/Ashland; Soliberine® [INCI: Buddleja Officinalis Flower Extract] marketed by Greentech; Citrustem™ [INCI: Xanthan Gum, Gluconolactone, Calcium Gluconate] or Lipout™ [INCI: Caprylic/Capric Glycerides, Plankton Extract] marketed by Provital; Dermcom™ [INCI: Acacia Senegal Gum, Crocus Chrysanthus Bulb Extract] marketed by Mibelle Biochemistry; Novhyal® Biotech G [INCI: Disodium Acetyl Glucosamine Phosphate] marketed by Induchem/Givaudan; Sphingokine® NP [INCI: Caprooyl Phytosphingosine] marketed by Evonik; Syn®-TC [INCI: Tetradecyl Aminobutyroylvalylaminobutyric Urea Trifluoroacetate, Palmitoyl Tripeptide-5, Palmitoyl Dipeptide-5 Diaminobutyroyl Hydroxythreonine] marketed by Pentapharm/DSM; Body³ Complex™ [INCI: Bentonite, Butyrospermum Parkii (Shea) Nut Extract, Persea Gratissima (Avocado) Fruit Extract], Progeline™ [INCI: Dextran, Trifluoroacetyl Tripeptide-2], Elix-IR™ [INCI: Polygonum Aviculare Extract] or Adipofill'in™ [INCI: Ornithine, Phospholipids, Glycolipids] marketed by Lucas Meyer Cosmetics/Unipex; Actiporine™ 8G [Jania Rubens Extract] marketed by Codif; ProSynergen™ DF [INCI: Lactobacillus/Ulkenia amoeboidea Ferment Extract Filtrate] marketed by Lonza; minerals such as calcium, among others, retinoids and their derivatives, isoflavonoids, carotenoids, in particular lycopene, pseudodipeptides, retinoids and their derivatives such as retinol or retinyl palmitate, among others, or heparinoids, among others.

The cosmetic or pharmaceutical composition can further comprise an anti-wrinkle and/or antiaging agent selected, for example and not restricted to, from the group consisting of the extracts or hydrolyzed extracts of Vitis vinifera, Rosa canina, Curcuma longa, Theobroma cacao, Ginkgo biloba, Leontopodium alpinum or Dunaliella salina among others, Matrixyl® [INCI: Palmitoyl Pentapeptide-4], Matrixyl® 3000® [INCI: Palmitoyl Tetrapeptide-7, Palmitoyl Oligopeptide], Matrixyl® Synthe'6™ [INCI: Hydroxypropyl Cyclodextrin, Palmitoyl Tripeptide-38], Essenskin™ [INCI: Calcium Hydroxymethionine], Renovage™ [INCI: Teprenone], Resistem™ [INCI: Globularia Cordifolia Ferment], Dermaxyl® [INCI: Palmitoyl Oligopeptide], Beautifeye™ [INCI: Albizia Julibrissin Bark Extract, Darutoside], Meiritage™ [INCI: Astragalus Membranaceus Root Extract, Atractylodes Macrocephala Root Extract, Bupleurum falcatum Root Extract], Senestem™ [INCI: Plantago Lanceolata Leaf Extract], Venuceane™ [INCI: Thermus Thermophilus Ferment] or Majestem™ [INCI: Leontopodium alpinum callus Culture Extract] marketed by Sederma/Croda; Vialox® [INCI: Pentapeptide-3], Syn® Ake® [INCI: Dipeptide Diaminobutyroyl Benzylamide Diacetate], Syn®-Coll [INCI: Palmitoyl Tripeptide-5], Phytaluronate™ [INCI: Locust Bean (Ceratonia Siliqua) Gum], Preregen® [INCI: Glycine soja (Soybean) Protein, Oxido Reductases], Syn®-TC [INCI: Tetradecyl Aminobutyroylvalylaminobutyric Urea Trifluoroacetate, Palmitoyl Tripeptide-5, Palmitoyl Dipeptide-5 Diaminobutyroyl Hydroxythreonine], Syn-Star™ [INCI: Dipeptide Diaminobutyroyl Benzylamide Diacetate] or Regu®-Scence [INCI: Asparagus Officinalis Stem Extract, Gluconolactone, Calcium Gluconate] marketed by Pentapharm/DSM; Myoxinol™ [INCI: Hydrolyzed Hibiscus esculentus Extract], Syniorage™ [INCI: Acetyl Tetrapeptide-11], Dermican™ [INCI: Acetyl Tetrapeptide-9], DN AGE™ LS [INCI: Cassia Alata Leaf Extract], EquiStat™ [INCI: Pyrus Malus Fruit Extract, Glycine soja Seed Extract], Juvenesce™ [INCI: Ethoxydiglycol, Caprylic Triglyceride, Retinol, Ursolic Acid, Phytonadione, Ilomastat], Shadownyl™ [INCI: Algae Extract], Epigenist™ [INCI: Voandzeia Subterranea Seed Extract, Maltodextrin], LOX-AGE™ [INCI: Cichorium Intybus (Chicory) Leaf Extract] or Perlaura™ [INCI: Polygonum Bistorta Root Extract] marketed by BASF; Algisum C® [INCI: Methylsilanol Mannuronate], Hydroxyprolisilane CN® [INCI: Methylsilanol Hydroxyproline Aspartate] or Exage™ [INCI: Imidazolylethyl Diaminopropanamide] marketed by Exsymol; Argireline® [INCI: Acetyl Hexapeptide-8], SNAP-7 [INCI: Acetyl Heptapeptide-4], SNAP-8™ [INCI: Acetyl Octapeptide-3], Leuphasyl® [INCI: Pentapeptide-18], Inyline® [INCI: Acetyl Hexapeptide-30], Aldenine® [INCI: Hydrolyzed Wheat Protein, Hydrolyzed Soy Protein, Tripeptide-1], Preventhelia® [INCI: Diaminopropionoyl Tripeptide-33], Decorinyl® [INCI: Tripeptide-10 Citrulline], Decorinol® [INCI: Tripeptide-9 Citrulline], Trylagen® [INCI: Pseudoalteromonas Ferment Extract, Hydrolyzed Wheat Protein, Hydrolyzed Soy Protein, Tripeptide-10 Citrulline, Tripeptide-1], Eyeseryl® [INCI: Acetyl Tetrapeptide-5], Peptide AC29 [INCI: Acetyl Tripeptide-30 Citrulline], Relistase® [INCI: Acetylarginyltriptophyl Diphenylglycine], Thermostressine® [INCI: Acetyl Tetrapeptide-22], Lipochroman® [INCI: Dimethylmethoxy Chromanol], Chromabright® [INCI: Dimethylmethoxy Chromanyl Palmitate], Antarcticine® [INCI: Pseudoalteromonas Ferment Extract], dGlyage® [INCI: Lysine HCl, Lecithin, Tripeptide-9 Citrulline], Vilastene™ [INCI: Lysine HCl, Lecithin, Tripeptide-10 Citrulline], Hyadisine® [INCI: Pseudoalteromonas Ferment Extract], Hyanify™ [INCI: Saccharide Isomerate], Diffuporine® [INCI: Acetyl Hexapeptide-37], Silusyne® [INCI: Soybean (Glycine soja) Oil, Sorbitan Sesquioleate, Isohexadecane, Sodium Hyaluronate, Lauryldimonium Hydroxypropyl Hydrolyzed Soy Protein, Acetyl Hexapeptide-39], Adifyline® [INCI: Acetyl Hexapeptide-38], Uplevity™ [INCI: Acetyl Tetrapeptide-2], Juveleven™ [INCI: Acetyl Hexapeptide-51 Amide], Argirelox™ [INCI: Acetyl Hexapeptide-8, Pentapeptide-18], Seacode™ [INCI: Pseudoalteromonas Ferment Extract], Eyedeline™ [INCI: Plankton Extract] or Cellynkage™ [INCI: Saccharide Isomerate] marketed by Lipotec/Lubrizol; Kollaren® [INCI: Tripeptide-1, Dextran] marketed by Institut Européen de Biologie Cellulaire; Collaxyl® IS [INCI: Hexapeptide-9], Laminixyl IS™ [INCI: Heptapeptide], Orsirtine™ GL [INCI: Oryza Sativa (Rice) Extract], D'Orientine™ IS [INCI: Phoenix Dactylifera (Date) Seed Extract], Phytoquintescine™ [INCI: Einkorn (Triticum Monococcum) Extract], Quintescine™ IS [INCI: Dipeptide-4], Peptide Q10™ [INCI: Pentapeptide-34 Trifluoroacetate] or Telosense™ [INCI: Hydrolyzed Yeast Protein, Hydrolyzed Soy Protein] marketed by Vincience/ISP/Ashland; BONT-L-Peptide [INCI: Palmitoyl Hexapeptide-19] marketed by Infinitec Activos; Deepaline™ PVB [INCI: Palmitoyl hydrolyzed Wheat Protein], Sepilift® DPHP [INCI: Dipalmitoyl Hydroxyproline] or Ephemer™ [INCI: Caprylic/Capric Triglyceride, Undaria Pinnatifida Extract] marketed by Seppic; Gatuline® Expression [INCI: Acmella Oleracea Extract], Gatuline® In-Tense [INCI: Spilanthes Acmella Flower Extract], Gatuline® Age Defense 2 [INCI: Juglans Regia (Walnut) Seed Extract] or Gatuline® Renew [INCI: Cryptomeria Japonica Bud Extract] marketed by Gattefosse; Thalassine™ [INCI: Algae Extract] marketed by Biotechmarine; ChroNOline™ [INCI: Caprooyl Tetrapeptide-3] or Thymulen-4 [INCI: Acetyl Tetrapeptide-2] marketed by Atrium/Unipex Innovations; Ameliox™ [INCI: Carnosine, Tocopherol, Silybum marianum Fruit Extract], PhytoCellTec™ Malus Domestica [INCI: Malus Domestica Fruit Cell Culture], PhytoCellTec™ Symphytum [INCI: Isomalt, Symphytum Officinale Root Cell Culture, Lecithin], PhytoCellTec™ Nunatak® [INCI: Saponaria Pumila Callus Culture Extract, Isomalt, Lecithin], AnaGain™ [INCI: Pisum Sativum (Pea) Sprout Extract], Dermcom™ [INCI: Acacia Senegal Gum, Crocus Chrysanthus Bulb Extract], Snow Algae Powder [INCI: Chlamydocapsa Extract, Maltodextrin, Lecithin], RoyalEpigen™ P5 [INCI: Pentapeptide-48, Hydrogenated Lecithin, Butyrospermum Parkii (Shea) Butter] or Vin-upLift™ [INCI: Wine, Caesalpinia Spinosa Gum] marketed by Mibelle Biochemistry; Bioxilift™ [INCI: Pimpinella Anisum Extract], SMS Anti-Wrinkle® [INCI: Annona Squamosa Seed Extract] Vitagenyl® [INCI: Prunus persica (Peach) Leaf Extract], Epigenomyl® [INCI: Calendula officinalis Flower Extract], Eternaline® [INCI: Helichrysum Stoechas Extract] or Filmexel® [INCI: Kappaphycus Alvarezii Extract, Caesalpinia Spinosa Fruit Extract] marketed by Silab; Citrustem™ [INCI: Xanthan Gum, Gluconolactone, Calcium Gluconate], Melavoid™ [INCI: Boerhavia Diffusa Root Extract], Darkout™ [INCI: Hypoxis Rooperi Rhizome Extract, Caesalpinia Spinosa Gum], Linefill™ [INCI: Caprylic/Capric Glycerides, Dimethyl Isosorbide, Sesamum indicum (Sesame) Seed Extract] or Lingostem™ [INCI: Vaccinium Vitis-ldaea Fruit Extract] marketed by Provital; Novhyal® Biotech G [INCI: Disodium Acetyl Glucosamine Phosphate], Rubixyl® [INCI: Hexapeptide-47] or Tightenyl™ [INCI: Disodium Acetyl Glucosamine Phosphate, Sodium Glucuronate] marketed by Induchem/Givaudan; Sphingokine® NP [INCI: Caprooyl Phytosphingosine] marketed by Evonik; Effipulp® [INCI: Hydrolyzed Avocado Protein, Maltodextrin] or Neurovity® [INCI: Vitex Negundo Extract] marketed by Laboratoires Expanscience; Progeline™ [INCI: Dextran, Trifluoroacetyl Tripeptide-2], Adipofill'in™ [INCI: Ornithine, Phospholipids, Glycolipids], Elix-IR™ [INCI: Polygonum Aviculare Extract] or SWT-7™ [INCI: Swertia Chirata Extract] marketed by Lucas Meyer Cosmetics/Unipex; Amiperfect™ [INCI: Gaultheria Procumbens (Wintergreen) Leaf Extract] or Repulpami™ ER [INCI: Adansonia Digitata Pulp Extract, Hibiscus sabdariffa Flower Extract] marketed by Alban Muller; Liftonin®-Xpress [INCI: Hydroxypropyl Methylcellulose, Pullulan, Porphyridium Cruentum Extract] or Liftonin®-Xpert [INCI: Bulbine Frutescens Leaf Juice] marketed by Rahn; Actiporine™ 8G [Jania Rubens Extract], EPS Seafill™ [INCI: Plankton Extract] or Neuroguard™ [INCI: Hydrolyzed Algin] marketed by Codif; Celloxyl® [INCI: Uapaca Bojeri Leaf Extract] or Resistress® [INCI: Sophora Japonica Flower Extract] marketed by Solabia; ProSynergen™ DF [INCI: Lactobacillus/Ulkenia amoeboidea Ferment Extract Filtrate] marketed by Lonza; SymVital® AgeRepair [INCI: Zingiber Officinale (Ginger) Root Extract] marketed by Symrise; Cernilys® [INCI: Cedrus Atlantica Bark Extract] or Reverskin® [INCI: Phytosterols] marketed by Greentech; Clotholine® [INCI: Coumaroyl Methoxytryptamine, PEG-8 Caprylic/Capric Glycerides] marketed by Sollice Biotech; Fision™ WrinkleFix [INCI: Hydrolyzed Rice Protein, Hydrolyzed Pea Protein, Glycine, Proline, Hydrolyzed Sodium Hyaluronate] marketed by TRI-K Industries; antagonists of the Ca²⁺ channel such as and not restricted to, alverine, manganese or magnesium salts, certain secondary or tertiary amines, retinol and its derivatives, idebenone and its derivatives, Coenzyme Q10 and its derivatives, boswellic acid and its derivatives, GHK and its derivatives and/or salts, carnosine and its derivatives, DNA repair enzymes such as and not restricted to, photolyase or T4 endonuclease V, or chloride channel agonists among others, and/or mixtures thereof.

The cosmetic or pharmaceutical composition can further comprise an anti-inflammatory agent and/or analgesic selected, for example and not restricted to, from the group consisting of extract of madecassoside, extract of echinacea, amaranth seed oil, sandal wood oil, extract of peach tree leaf, extract of Aloe vera, Arnica montana, Artemisia vulgaris, Asarum maximum, Calendula officinalis, Capsicum, Centipeda cunninghamii, Chamomilla recutita, Crinum asiaticum, Hamamelis virginiana, Harpagophytum procumbens, Hypericum perforatum, Lilium candidum, Malva sylvestris, Melaleuca alternifolia, Origanum majorana, Origanum vulgare, Prunus laurocerasus, Rosmarinus officinalis, Salix alba, Silybum marianum, Tanacetum parthenium, Thymus vulgaris, Uncaria guianensis or Vaccinium myrtillus, omega-3 and omega-6 fatty acids, Neutrazen™ [INCI: Dextran, Palmitoyl Tripeptide-8] marketed by Atrium Innovations/Unipex Group; Delisens™ [INCI: Acetyl Hexapeptide-49] marketed by Lipotec/Lubrizol; Meliprene® [INCI: Dextran, Acetyl Heptapeptide-1] marketed by Institut Europeen de Biologie Cellulaire/Unipex Group; Skinasensyl™ [INCI: Acetyl Tetrapeptide-15] or Anasensyl™ [INCI: Mannitol, Ammonium Glycyrrhizate, Caffeine, Hippocastanum (Horse Chestnut) Extract] marketed by BASF; Calmosensine™ [INCI: Acetyl Dipeptide-1] marketed by Sederma/Croda; coenzyme Q10 or alkyl glyceryl ethers, among others, or mixtures thereof.

The cosmetic or pharmaceutical composition can further comprise an agent capable of filtering UV and IRA rays, for example and not restricted to, from the group consisting of photoprotectors of an organic or mineral nature active against ultraviolet A and/or B rays such as substituted benzotriazoles, substituted diphenyl acrylates, organic complexes of nickel, umbelliferone, urocanic acid, derivatives of biphenyl, e-stilbene, 3-benzylidene camphor, and their derivatives such as 3-(4-methylbenzylidene)camphor; derivatives of 4-aminobenzoic acid, 2-ethylhexyl 4-(dimethylamino)benzoate, 2-octyl 4-(dimethylamino)benzoate and amyl 4-(dimethylamino)benzoate; cinnamic acid esters, such as 2-ethylhexyl 4-methoxycinamate or diethylamino hydroxybenzoyl hexyl benzoate, propyl 4-methoxycinnamate, isoamyl 4-methoxycinnamate, 2-ethylhexyl 2-cyano-3,3-phenyl cinnamate (octocrylenes); salicylic acid esters, such as 2-ehtylhexyl salicylate, 4-isopropyl benzyl salicylate, homomenthyl salicylate; derivatives of benzophenone, such as 2-hydroxy-4-methoxybenzophenone, 2-hydroxy-4-methoxy-4′-methylbenzophenone, 2,2′-dihydroxy-4-methoxybenzophenone; benzalmalonic acid esters, such as di-2-ethylhexyl 4-methoxybenzalmalonate; derivatives of triazine, such as 2,4,6-trianilino, p-carbo-2′-ethyl-1′-hexyloxy-1,3,5-triazine, octyl triazone or dioctylbutamidotriazones; propane-1,3-diones, such as 1-(4-tert-butylphenyl)-3-(4′-methoxyphenyl)propane-1,3-dione; derivatives of ketotricyclo(5.2.1.0)decane; 2-phenylbenzimidazole-5-sulfonic acid; derivatives of benzophenone sulfonic acid, such as 2-hydroxy-4-methoxybenzofenone-5-sulfonic acid and its salts; 4-(2-oxo-3-bornylidenemethyl)benzenesulfonic acid, derivatives of benzoylmethane, such as benzoylmethane 2-methyl-5-(2-oxo-3-bornylidene)sulfonic acid, such as 1-(4′-tert-butylphenyl)-3-(4′-methoxyphenyl)propane-1,3-dione, 4-tert-butyl-4′-methoxydibenzoylmethane, 1-phenyl-3-(4′-isopropylphenyl)-propane-1,3-dione, compounds of enamine, anthranilates, silicones, derivatives of benzimidazole, imidazolines, derivatives of benzo allyl, Chromabright® [INCI: Dimethylmethoxy Chromanyl Palmitate] or Preventhelia® [INCI: Diaminopropionoyl Tripeptide-33] both marketed by Lipotec, metal oxides such as zinc, titanium, iron, zirconium, silicon, manganese, aluminum and cerium oxides; silicates, talc, barium sulfate, zinc stearate, carbon nanotubes and/or mixtures thereof.

The cosmetic or pharmaceutical composition can further comprise a reactive carbonyl species scavenger, free radical scavengers and/or anti-glycation agent, detoxifying agent, antioxidant and/or anti-pollution agent selected, for example and not restricted to, from the group consisting of carnosine and its derivatives, GHK [INCI: Tripeptide-1] and its salts and/or derivatives, Quintescine™ IS [INCI: Dipeptide-4] marketed by Vincience/ISP/Ashland; Melitane™ [INCI: Dextran, Acetyl Hexapeptide-1], Homeoxy™ [INCI: Enteromorpha Compressa, Palmaria Palmata Extract] or Lanatellis™ [INCI: Chrysanthellum indicum Extract, Camellia Sinensis Leaf Extract] marketed by Atrium Innovations/Lucas Meyer Cosmetics; Protectan™ [INCI: Lactococcus Ferment Lysate] marketed by CLR; Phycosaccharide™ [INCI: Hydrolyzed Algin, Magnesium Sulfate, Manganese Sulfate] or Algowhite™ [INCI: Ascophyllum Nodosum Extract] marketed by Codif; Preregen™ [INCI: Glycine soja (Soybean) Protein, Oxido Reductases], Edelweiss GC™ [INCI: Leontopodium alpinum Extract], Lipogard™ [INCI: Squalane, Ubiquinone], Nectapure™ [INCI: Buddleja Davidii Extract, Thymus vulgaris Extract], Alpaflor Nectapure™ [INCI: Buddleja Davidii Extract, Thymus vulgaris Extract] or Dismutin-BT™ [INCI: Superoxide Dismutase] marketed by Pentapharm/DSM; TEGO Turmerone™ [INCI: Curcuma longa Extract] marketed by Evonik Goldschmidt; Hierogaline™ [INCI: Triticum vulgare (Wheat) Germ Oil Unsaponifiables, Sesamum indicum (Sesame) Oil Unsaponifiables] marketed by Expanscience Laboratoires; Glistin™ [INCI: Glutamylamidoethyl Indole], Glutrapeptide™ [INCI: Pyroglutamylamidoethyl Indole], Algisium™ C [INCI: Methylsilanol Mannuronate], Silysin™ C [INCI: Silanetriol Lysinate], Exsy-Arl™ [INCI: Prolinamidoethyl Imidazole] or OTZ-10™ [INCI: Oxothiazolidine] marketed by Exsymol; Gatuline™ Skin-Repair Bio [INCI: Onopordum Acanthium Flower/Leaf/Stem Extract] marketed by Gattefosse; Preventhelia® [INCI: Diaminopropionoyl Tripeptide-33], Aldenine® [INCI: Hydrolyzed Wheat Protein, Hydrolyzed Soy Protein, Tripeptide-1], Lipochroman® [INCI: Dimethylmethoxy Chromanol], Thermostressine® [INCI: Acetyl Tetrapeptide-22], Bodyfensine® [INCI: Acetyl Dipeptide-3 Aminohexanoate] or Pollushield™ [INCI: Diisopropyl Adipate, Lecithin, Acrylic Acid/Acrylamidomethyl Propane Sulfonic Acid Copolymer, Dimethylmethoxy Chromanol, Xanthan Gum] marketed by Lipotec/Lubrizol; Setiline™ [INCI: Hydrolyzed Trigonella Foenum-Graecum Seed Extract] marketed by Greentech; Sunactyl™ [INCI: Mannitol, Pisum Sativum Extract, Histidine HCl, Arginine, Cyclodextrin, Dextrin, Yeast Extract, Acetyl Tyrosine, Pyridoxine HCl, Khaya Senegalensis Bark Extract, Nicotinamide, Adenine Dinucleotide, Disodium Succinate, Aspartic Acid], Imidinyl™ [INCI: Tamarindus Indica Seed Polysaccharide], Phystrogene™ [INCI: Malva sylvestris (Mallow) Extract, Xanthan Gum] or Purisoft™ [INCI: Moringa Pterygosperma Seed Extract] marketed by BASF; AquaCacteen™ [INCI: Opuntia Ficus Indica Stem Extract], Trimoist™ (KMF) [INCI: Sodium Stearoyl Lactylate, Cetyl Alcohol, Vegetable Oil, Tocopheryl Acetate, Glycine soja Sterol, Sodium Lactate, Sodium Carboxymethyl Betaglucan, Carnosine], MelanoBronze™ [INCI: Vitex agnus castus Extract (Monk's pepper berries extract (phyto-endorphins)), Acetyl Tyrosine], CM-Glucan [INCI: Sodium Carboxymethyl Betaglucan], SunActin™ [INCI: Helianthus annuus (Sunflower) Sprout Extract, Tocopherols, Lecithin], GSP-T™ skin [INCI: PEG-40 Hydrogenated Castor Oil, Vitis vinifera (Grape) Seed Extract] or Detoxophane™ [INCI: Lepidium Sativum Sprout Extract, Lecithin] marketed by Mibelle Biochemistry; Bacocalmine™ [INCI: PEG-8, Bacopa Monnieri Extract, Hydroxyethylcellulose], Kombuchka™ [INCI: Saccharomyces/Xylinum Black Tea Ferment, Hydroxyethyl Cellulose] or Prodizia™ [INCI: Albizia Julibrissin Extract] marketed by Sederma/Croda; Extramel™ C [INCI: Hydroxypropyltrimonium Maltodextrin Crosspolymer, Cucumis Melo (Melon) Fruit Extract] marketed by Seppic; Defensine™ [INCI: Triticum vulgare Germ Extract] or Antiglyskin™ [INCI: Helianthus annuus Seed Extract] marketed by Silab; ATP 23 [INCI: Azeloyl Tetrapeptide-23] marketed by Sinergia; Glycofilm™ [INCI: Biosaccharide Gum-4] marketed by Solabia.

The compositions of the invention may be for use in any of the applications or uses discussed above under the heading “Applications”.

The invention is illustrated by the following non-limiting examples.

### EXAMPLES

### General Methodology

All reagents and solvents are of synthesis quality and are used without additional treatment.

### Abbreviations

The abbreviations used for amino acids follow the 1983 IUPAC-IUB Joint Commission on Biochemical Nomenclature recommendations outlined in Eur. J. Biochem. (1984) 138:9-37.

(R), resin; 2-ClTrt-(R), 2-chlorotrityl resin; Ac, acetyl; AcOH, acetic acid; Ala, alanine; AM, 2-[4-aminomethyl-(2,4-dimethoxyphenyl)] phenoxyacetic acid; Boc, tert-butyloxycarbonyl; DCM, dichloromethane; DIEA, N,N′-diisopropylethylamine; DIPCDI, N,N′-diisopropylcarbodiimide; DMF, N,N-dimethylformamide; ESI-MS, electrospray ionization mass spectrometry; Fmoc, 9-flluorenylmethyloxycarbonyl; HOBt, 1-hydroxybenzotriazole; HPLC, high performance liquid chromatography; Ile, isoleucine; KOH, potassium hydroxide; Leu, leucine; MBHA, p-methylbenzhydrylamine; MeCN, acetonitrile; MeOH, methanol; Met, methionine; Myr, myristoyl; Palm, palmitoyl; Phe, phenylalanine; tBu, tert-butyl; TFA, trifluoroacetic acid; Trp, tryptophan; Tyr, tyrosine; Val, valine.

### Chemical Synthesis

All synthetic processes are carried out in polypropylene syringes fitted with porous polyethylene discs. All the reagents and solvents are synthesis quality and are used without any additional treatment. The solvents and soluble reagents are removed by suction. The Fmoc group is removed with piperidine-DMF (2:8, v/v) (1×1 min, 1×5 min, 5 mL/g resin) [Lloyd-Williams P. et al. (1997) “Chemical Approaches to the Synthesis of Peptides and Proteins” CRC, Boca Raton (FL, USA)]. Washes between stages of deprotection, coupling, and, again, deprotection, are carried out with DMF (3×1 min) each time using 10 mL solvent/g resin. Coupling reactions are performed with 3 mL solvent/g resin. The control of the couplings is performed by carrying out the ninhydrin test [Kaiser, E., et al., Anal. Biochem., (1970), 34: 595-598] or chloranil test [Christensen T., Acta Chem. Scand., (1979), 33B, 763-766]. All synthetic reactions and washes are carried out at 25° C.

HPLC chromatographic analysis is carried out with Shimadzu equipment (Kyoto, Japan) using a reversed-phase column thermostatized at 300° C. (50×4.6 mm, Kromasil C18, 3.5 μm, Akzo Nobel, Sweden). The elution is carried out using a gradient of acetonitrile (+0.07% TFA) in water (+0.1% TFA) at a flow rate of 1.6 mL/min and detection is carried out at 220 nm. The electrospray ionization mass spectrometry is carried out in a WATERS Alliance ZQ™ 2000 detector using a mixture of MeCN:H₂O 4:1 (+0.1% TFA) as the mobile phase and a flow rate of 0.3 mL/min.

### Example 1

Obtaining H-AA₃-O-2-ClTrt-(R), Wherein AA₃ is L-Tyr or L-Phe

Weights have been normalized. 3.2 mmol (1 equiv) of Fmoc-L-Tyr(tBu)-OH or 1.6 mmol (1 equiv) of Fmoc-L-Phe-OH, dissolved in 20 or 10 mL of DCM, to which is added 0.83 equiv of DIEA, is coupled onto dry 2-chlorotrityl resin (3.2 mmol or 1.6 mmol) with a functionalization 1.6 mmol/g. The mixture is stirred for 5 min, after which 1.63 equiv of DIEA are added. The mixture is left to react for 40 min. The remaining chloride groups are blocked by treatment with 1 or 2 mL of MeOH. The resin is then washed and the N-terminal Fmoc group is deprotected as described in the general methods.

Obtaining Fmoc-Wₘ—Xₙ-AA₁-AA₂-AA₃-Yₚ—Z_{q}—O-2-ClTrt-(R), Wherein AA₁ is L-Tyr; AA₂ is L-Ile or L-Leu; AA₃ is L-Tyr or L-Phe; and n, m, p and q are Each 0.

Weights have been normalized. 220 mg H-L-Tyr(tBu)-2-Cl-Trt resin with a functionalization of 1.03 mmol/g (0.23 mmol) or 500 mg H-L-Phe-2-Cl-Trt resin with a functionalization of 0.92 mmol/g (0.46 mmol) is washed as described in the general methods. Following the protocols described, 5 equiv of Fmoc-Leu-OH or Fmoc-Ile-OH is coupled onto the peptidyl resins in the presence of 5.5 equiv of DIPCDI and 5 equiv HOBt using DMF as a solvent for 90 minutes.

The N-terminal Fmoc group is deprotected as described in the general methods and 5 equiv of Fmoc-L-Tyr(tBu)-OH is coupled onto the peptidyl resins in the presence of 5.5 equiv of DIPCDI and 5 equiv of HOBt using DMF as a solvent for 90 minutes.

### Example 2

Obtaining Fmoc-Wₘ—Xₙ-AA₁-AA₂-AA₃-Yₚ—Z_{q}-AM-MBHA-(R), Wherein AA₁ is L-Tyr- or L-Trp; AA₂ is L-Ile, L-Leu, L-Met or L-Val; AA₃ is L-Phe, L-Trp or L-Tyr; and n, m, p and q are Each 0.

Weights have been normalized. 350 mg (0.18 mmol) or 250 mg (0.13 mmol) of Fmoc-AM-MBHA resin with a functionalization of 0.52 mmol/g is treated with piperidine:DMF according to the described general protocol in order to remove the Fmoc group. 5 equiv of Fmoc-L-Phe-OH or Fmoc-L-Trp(Boc)-OH, or Fmoc-L-Tyr(tBu)-OH is incorporated onto the deprotected resin in the presence of 5.5 equiv of DIPCDI and 5 equiv of HOBt using DMF as a solvent for 1 hour.

The resin is then washed as described in the general methods and the deprotection treatment of the Fmoc group is repeated to couple the next amino acid. Following the previously described protocols, 5 equiv of Fmoc-L-Ile-OH, Fmoc-L-Leu-OH, Fmoc-L-Met-OH or Fmoc-L-Val-OH; and subsequently 5 equiv of Fmoc-L-Trp(Boc)-OH or Fmoc-L-Tyr(tBu)-OH; are sequentially coupled in the presence of 5 equiv of HOBt and 5.5 equiv of DIPCDI in each coupling step.

After the synthesis, the peptidyl resin is washed with DCM (3×1 min).

### Example 3

Obtaining Fmoc-Wₘ—Xₙ-AA₁-AA₂-AA₃-Yₚ—Z_{q}-AM-MBHA-(R), Wherein X is L-Phe; AA₁ is L-Tyr; AA₂ is L-Val; AA₃ is L-Tyr; Y is L-Phe; m and q are Each 0; and n and p are Each 1.

400 mg of Fmoc-AM-MBHA resin with a functionalization of 0.52 mmol/g (0.21 mmol) is treated with piperidine:DMF according to the described general protocol in order to remove the Fmoc group. 403 mg of Fmoc-L-Phe-OH, (1.0 mmol; 5 equiv) is incorporated onto the deprotected resin in the presence of 176 μL of DIPCDI (1.15 mmol; 5.5 equiv) and 159 mg of HOBt (1.0 mmol; 5 equiv) using DMF as a solvent for 1 hour.

The resin is then washed as described in the general methods and the deprotection treatment of the Fmoc group is repeated to couple the next amino acid. Following the previously described protocols 478 mg Fmoc-L-Tyr(tBu)-OH (1.0 mmol, 5 equiv); and subsequently 353 mg of Fmoc-L-Val-OH (1.0 mmol, 5 equiv); 478 mg of Fmoc-L-Tyr(tBu)-OH; and 353 mg of Fmoc-L-Phe-OH (1.0 mmol, 5 equiv) are sequentially coupled in the presence of 159 mg of HOBt (1.0 mmol, 5 equiv) and 176 μL of DIPCDI (1.15 mmol; 5.5 equiv) in each coupling step.

After the synthesis, the peptidyl resin is washed with DCM (3×1 min).

### Example 4

### General Process for Removal of Fmoc N-Terminal Protective Group.

The N-terminal Fmoc group of the peptidyl resins obtained in examples 1, 2 and 3 is deprotected as described in the general methods (20% piperidine in DMF, 1×1 min+1×5 min). The peptidyl resins are washed with DMF (5×1 min), DCM (3×1 min), diethyl ether (3×1 min) and dried under vacuum.

### Example 5

Process for Introducing the R₁ Palmitoyl Group onto the Peptidyl Resins Obtained in Example 4.

233 mg of palmitic acid (0.9 mmol; 5 equiv), pre-dissolved in DMF (1 mL), is added onto 0.18 mmol of each of the peptidyl resin obtained in Example 4, in the presence of 139 mg of HOBt (0.9 mmol; 5 equiv) and 154 μL of DIPCDI (0.99 mmol; 5.5 equiv). The mixture is allowed to react for 3 hours, after which the resin is washed with DMF (3×1 min), DCM (3×1 min), diethyl ether (3×1 min) and is dried under vacuum.

### Example 6

Process for Introducing the R₁ Myristoyl Group onto the Peptidyl Resins Obtained in Example 4.

208 mg of myristic acid (0.9 mmol; 5 equiv), pre-dissolved in DMF (1 mL), is added onto 0.18 mmol of each of the peptidyl resins obtained in Example 4, in the presence of 1392 mg of HOBt (0.9 mmol; 5 equiv) and 154 μL of DIPCDI (0.99 mmol; 5.5 equiv). The mixture is allowed to react for 3 hours, after which the resin is washed with DMF (3×1 min), DCM (3×1 min), diethyl ether (3×1 min) and is dried under vacuum.

### Example 7

Process for Introducing the R₁ Acetyl Group onto the Peptidyl Resins Obtained in Example 4.

0.13 mmol of each of the peptidyl resins obtained in Example 4 is treated with 25 equiv of acetic anhydride in the presence of 25 equiv of DIEA using 2 mL of DMF as a solvent. The mixture is allowed to react for 30 min, after which the resin is washed with DMF (3×1 min), DCM (3×1 min), diethyl ether (3×1 min) and is dried under vacuum.

### Example 8

Cleavage Process from the Polymeric Support of the Peptidyl Resins Obtained in Examples 4, 5, 6 and 7.

264 mg of each of the dried peptidyl resins obtained in Examples 4, 5, 6 and 7 are treated with 2.5 mL of TFA:H₂O (95:5, v/v) for 2 hours at room temperature under stirring. The filtrate is collected onto 17 mL of cold diethyl ether, then it is filtered through a polypropylene syringe fitted with porous polyethylene discs and washed 5 times with 10 mL diethyl ether. The final precipitate is dried under vacuum.

HPLC analyses of the obtained peptides in gradients of MeCN (+0.07% TFA) in H₂O (+0.1% TFA) show a purity exceeding 80% in all cases. The identity of the peptides obtained is confirmed by ESI-MS.

### Example 9

Cleavage process from the polymeric support and functionalization with R₂ substituted amine: Obtaining H—Wₘ—Xₙ-AA₁-AA₂-AA₃-Yₚ—Z_{q}—NH—(CH₂)₅—CH₃, wherein AA₁ is L-Tyr; AA₂ is L-Ile or L-Val; AA₃ is L-Phe or L-Tyr; and n, m, p and q are each 0.

The peptides H—Wₘ—Xₙ-AA₁-AA₂-AA₃-Yₚ—Z_{q}—OH with fully protected side chains are obtained by treating 596 mg of each of the peptidyl resins H—Wₘ—Xₙ-AA₁-AA₂-AA₃-Yₚ—Z_{q}—O-2-ClTrt-(R) of Example 4, which are previously desiccated under vacuum in the presence of KOH, with 4.2 mL AcOH for 2 hours. Liquid phase is separated through filtration. The filtrate is collected and the resin is then washed with 3 mL AcOH (1×1 min). All the collected liquid phases are combined and lyophilized.

100 mg of each of the obtained crude peptides is weighed in a flask and 3 equiv of Hexylamine. HOBt and 15 mL of anhydrous DMF is added. 2 equiv of DIPCDI is added, and left to react under magnetic stirring at 47° C. The reaction is monitored by HPLC until disappearance of the initial products, and is complete after 2-4 hours. Solvents are evaporated to dryness and co-evaporated twice with DCM. The obtained residue is dissolved in 25 mL of a mixture of TFA:H₂O (95:5, v/v) and left to react for 2 hours at room temperature. 175 mL of cold diethyl ether are added, the solvents are evaporated under reduced pressure and two additional co-evaporations with diethyl ether are carried out. The residue is dissolved in a mixture of 50% MeCN in H₂O (v/v) and lyophilized.

HPLC analyses of the obtained peptides in gradients of MeCN (+0.07% TFA) in H₂O (+0.1% TFA) show a purity exceeding 80% in all cases. The identity of the peptides obtained is confirmed by ESI-MS.

### Example 10

Cleavage process from the polymeric support and functionalization with R₂ substituted amine: Obtaining H—Wₘ—Xₙ-AA₁-AA₂-AA₃-Yₚ—Z_{q}—NH—(CH₂)₁₅—CH₃, wherein AA₁ is L-Tyr; AA₂ is L-Ile or L-Val; AA₃ is L-Phe or L-Tyr; and n, m, p and q are each 0.

The peptides H—Wₘ—Xₙ-AA₁-AA₂-AA₃-Yₚ—Z_{q}—OH with fully protected side chains are obtained by treating 596 mg of each of the peptidyl resins H—Wₘ—Xₙ-AA₁-AA₂-AA₃-Yₚ—Z_{q}—O-2-ClTrt-(R) of Example 4, which are previously desiccated under vacuum in the presence of KOH, with 4.2 mL AcOH for 2 hours. The liquid phase is separated through filtration. The filtrate is collected and the resin is then washed with 3 mL AcOH (1×1 min). All the collected liquid phases are combined and lyophilized.

100 mg of each of the obtained crude peptides is weighed in a flask and 3 equiv of Hexadecylamine. HOBt and 15 mL of anhydrous DMF are added. 2 equiv of DIPCDI is added, and left to react being magnetically stirred at 47° C. The reaction is monitored by HPLC until disappearance of the initial products, and is complete after 2-4 hours. Solvents are evaporated to dryness and co-evaporated twice with DCM. The obtained residue is dissolved in 25 mL of a mixture of TFA:H₂O (95:5, v/v) and left to react for 2 hours at room temperature. 175 mL of cold diethyl ether is added, the solvents are evaporated under reduced pressure and two additional co-evaporations with diethyl ether are carried out. The residue is dissolved in a mixture of 50% MeCN in H₂O (v/v) and lyophilized.

HPLC analyses of the obtained peptides in gradients of MeCN (+0.07% TFA) in H₂O (+0.1% TFA) show a purity exceeding 80% in all cases. The identity of the peptides obtained is confirmed by ESI-MS.

### Example 11

### Study on the Reduction of the Percentage of Lipid Accumulation by a Fluorescent Assay for Determining Lipid Content Using Primary Human Subcutaneous Pre-Adipocyte Cells

The reduction of the percentage of lipid accumulation in human pre-adipocyte cells in a complete differentiation medium (basal control) is studied by treatment of the cells with the peptides of the invention. Quantification of lipid accumulation is evaluated with AdipoRed™ Assay Reagent (Lonza).

Human pre-adipocyte cells (Lonza) are seeded in quadruplicate (10.000 cells/well) on a 96-well plate and they are incubated on a complete growth medium (PGM2, Lonza) for 24 hours at 370° C. in a water-saturated atmosphere composed of 95% air and 5% CO₂.

The differentiation of pre-adipocytes into adipocytes is induced by changing the complete growth medium (PGM2, Lonza) for the complete differentiation medium (PDM2, Lonza). The cells are treated with the peptides of the invention at different concentrations prepared in complete differentiation medium. Caffeine (Sigma) at 0.2 mg/ml in PDM2 medium is used as a negative control of the lipid accumulation in the cells. Caffeine is the most widely used anti-cellulite cosmetic agent. After 8 days of treatment at 37° C. in an atmosphere composed of 95% air and 5% CO₂, lipid accumulation is measured for each peptide and concentration by fluorescence with an AdipoRed™ Assay (Lonza).

Manufacturer protocol is followed for the use of AdipoRed™ Assay Reagent (Lonza). In brief, the cells are washed and the reagent AdipoRed™ is added diluted (5/200). After being incubated for 15 minutes in darkness and at room temperature, the Relative Fluorescence Units (RFUs) are measured using a fluorescence plate reader (CLARIOstar™, BMG LABTECH), with excitation filter at 485 nm and emission filter at 572 nm.

The lipid accumulation percentage is calculated from these results with respect to the complete growth medium (PGM2 treatment as differentiation negative control, 0% lipid accumulation) and the complete differentiation medium (PDM2 treatment as basal control, 100% lipid accumulation). The results are calculated as the mean of all the independent experiments carried out in each case for each peptide and concentration assayed.

The obtained results are shown below in Table 3:

TABLE 3

| Sample | Concentration | | % Lipid Accumulation |
|---|---|---|---|
| Caffeine | 0.2 | mg/ml | 76.07 |
| H-Tyr-Val-Tyr-NH₂ | 0.1 | mg/ml | 32.65 |
| H-Tyr-Val-Tyr-NH₂ | 0.5 | mg/ml | 18.60 |
| H-Tyr-Val-Tyr-NH₂ | 1 | mg/ml | 15.65 |
| H-Tyr-Val-Tyr-OH | 0.1 | mg/ml | 35.14 |
| H-Tyr-Val-Tyr-OH | 0.5 | mg/ml | 33.55 |
| H-Tyr-Val-Tyr-OH | 1 | mg/ml | 22.66 |
| Ac-Tyr-Val-Tyr-NH₂ | 1 | mg/ml | 20.81 |
| Palm-Tyr-Val-Tyr-NH₂ | 0.1 | mg/ml | 86.17 |
| Myr-Tyr-Val-Tyr-NH₂ | 1 | mg/ml | 12.40 |
| H-Tyr-Val-Tyr-NHC₆H₁₃ | 1 | mg/ml | 30.79 |
| H-Tyr-Val-Tyr-NHC₁₆H₃₃ | 1 | mg/ml | 50.12 |
| Ac-Trp-Val-Tyr-NH₂ | 1 | mg/ml | 63.18 |
| H-Tyr-Ile-Tyr-NH₂ | 1 | mg/ml | 46.63 |
| H-Tyr-Ile-Tyr-NHC₆H₁₃ | 1 | mg/ml | 52.82 |
| H-Tyr-Ile-Tyr-NHC₁₆H₃₃ | 1 | mg/ml | 23.74 |
| Palm-Tyr-Leu-Tyr-NH₂ | 1 | mg/ml | 25.42 |
| Palm-Tyr-Leu-Tyr-OH | 0.005 | mg/ml | 79.98 |
| H-Tyr-Met-Tyr-NH₂ | 1 | mg/ml | 40.24 |
| Myr-Tyr-Val-Phe-NH₂ | 1 | mg/ml | 37.77 |
| H-Tyr-Val-Trp-NH₂ | 1 | mg/ml | 61.87 |
| H-Tyr-Ile-Phe-NH₂ | 1 | mg/ml | 47.45 |
| H-Tyr-Ile-Phe-NHC₆H₁₃ | 1 | mg/ml | 30.72 |
| H-Tyr-Ile-Phe-NHC₁₆H₃₃ | 1 | mg/ml | 40.61 |
| H-Tyr-Leu-Trp-NH₂ | 1 | mg/ml | 62.42 |
| H-Tyr-Val-Tyr-Phe-NH₂ | 1 | mg/ml | 86.68 |
| H-Tyr-Val-Tyr-Trp-NH₂ | 1 | mg/ml | 90.32 |
| H-Phe-Tyr-Val-Tyr-Phe-NH₂ | 1 | mg/ml | 64.31 |
| H-Phe-Trp-Tyr-Val-Tyr-NH₂ | 0.005 | mg/ml | 55.99 |
| H-Tyr-Val-Tyr-Phe-Trp-NH₂ | 1 | mg/ml | 76.47 |

The results show that the peptides of the invention are able to reduce the percentage of lipid accumulation in primary human subcutaneous pre-adipocytes under the test conditions.

### Example 12

Study of the Decrease in the Relative Level of Nocturnin with an ELISA (Enzyme-Linked Immunosorbent Assay) Colorimetric Test, Using a Primary Human Subcutaneous Pre-Adipocyte Cell Line.

Nocturnin, a rhythmic gene so called because its mRNA is transcribed at highest levels in the early night, controls specific circadian pathways related to lipid uptake and/or utilization. Cellulite results from an excessive fat storage in the adipose tissue which exerts considerable pressure on the surrounding skin tissue and creates a dimpled irregular appearance. Therefore, products that inhibit nocturnin protein levels may be good candidates for cellulite treatment during night.

The decrease in the relative level of nocturnin protein in human pre-adipocytes, cells that form adipose tissue, in a complete differentiation medium (basal condition) is studied when treating the peptides of the invention.

Human pre-adipocytes are seeded (10,000 cells/well, 4 wells per condition) on a 96-well plate and incubated on a complete growth medium (PGM2, LONZA) for 24 hours at 37° C. at an atmosphere with a CO₂ level of 5%.

After incubation, the cells are treated with the peptides at 0.1, 0.5 and 1 mg/ml and its respective vehicles prepared in a complete differentiation medium (PDM2, LONZA). After 6 days of treatment at an atmosphere with 37° C. and a 5% CO₂ level, the nocturnin level is measured using an ELISA test, and the total protein concentration is measured using a BCA (Bicinchoninic Acid) assay. For each concentration, the ELISA results are normalized with the BCA results. The relative nocturnin level is calculated from these results with respect to the basal condition (cells treated with complete differentiation medium). The values represent the mean of 3 independent experiments.

Measurement of Nocturnin Level (CCRN4L) with the ELISA (Enzyme-Linked Immunosorbent Assay) Test

The manufacturer protocol of the ELISA kit for Carbon Catabolite Repression 4 Like Protein (CCRN4L) (Uscn Life Science Inc) is followed. In brief, the cells are lysed and the cell extracts are incubated on a 96-well plate coated with an anti-body specific to CCRN4L. Afterwards, the wells are coated with an antibody specific to CCRN4L conjugated with Biotin. Next, the Avidin conjugated with the enzyme Horseradish Peroxidase (HRP) is added to the plate. Finally, 3,3′,5,5′-Tetramethylbenzidine substrate is added to the plate and a change in color is observed proportionally to the quantity of nocturnin in each concentration. Lastly, a sulfuric acid solution is added to stop the enzyme-substrate reaction and quantify the color change with an absorbance reader (CLARIOstar™, BMG LABTECH) at 450 and 570 nm.

### Determination of Total Protein by BCA Assay (Bicinchoninic Acid)

The manufacturer protocol of the BCA Protein assay kit (Pierce) is followed. The total protein concentration of the lysed cells is measured via a colorimetric reaction using a protein standard. In brief, the standard and the samples are distributed onto 96-well plates. The plate is then incubated with the reagent of the kit and finally the change in color is measured with the absorbance reader (CLARIOstar, BMG LABTECH) at 570 nm.

The obtained results are shown below in Table 4:

TABLE 4

| | | Nocturnin level with respect |
|---|---|---|
| Peptide | Concentration | to basal condition (%) |
| H-Tyr-Val-Tyr-NH₂ | 0.1 mg/mL | 0.76 |
| PEP-1-NH₂ | 0.5 mg/mL | 0.62 |
| | 1 mg/mL | 0.48 |
| H-Tyr-Val-Tyr-OH | 0.1 mg/mL | 0.67 |
| PEP-1 | 0.5 mg/mL | 0.87 |
| | 1 mg/mL | 0.83 |

The results show that the peptides of the invention reduce the relative level of nocturnin at all the concentrations studied, and a dose-response relationship is observed for PEP-1-NH₂.

### Example 13

Preparation of an Emulsion Comprising Peptide H-Tyr-Val-Tyr-NH₂ (PEP-1-NH₂)

In an appropriate container the peptide H-Tyr-Val-Tyr-NH₂ (PEP-1-NH₂) is dissolved in water [INCI: WATER (AQUA)] (phase A1), and then a mixture of the ingredients of phase A2 (2-phenoxyethanol [INCI: PHENOXYETHANOL], Structure® XL [INCI: HYDROXYPROPYL STARCH PHOSPHATE], Zemea™ [INCI: PROPANEDIOL], Amigel® [INCI: SCLEROTIUM GUM] and sodium hyaluronate [INCI: SODIUM HYALURONATE]; see Table 5), which have been pre-mixed in a separate recipient, is introduced. The resulting mixture is heated at 70° C. while stirring gently and then Cola®Fax CPE-K [INCI: POTASSIUM CETYL PHOSPHATE] is added (phase A3).

In another recipient, the components of phase B: Schercemol™ DIS Ester [INCI: DIISOPROPYL SEBACATE] and Montanov™ 68 [INCI: CETEARYL ALCOHOL; CETEARYL GLUCOSIDE] are introduced, heating them at 80° C. and stirring the mixture. Phase B is slowly introduced over phase A while intense stirring.

With the mixture at 70-80° C., 3 microfluidization cycles are carried out with an inlet pressure of 80 bar and an outlet pressure of 15000 bar.

TABLE 5

| Phase | INGREDIENT | % weight |
|---|---|---|
| A1 | WATER (AQUA) | q.s.100 |
| A1 | H-Tyr-Val-Tyr-NH₂ (PEP-1-NH₂) | 0.0547 |
| A2 | PHENOXYETHANOL | 2.7382 |
| A2 | HYDROXYPROPYL STARCH PHOSPHATE | 0.6570 |
| A2 | PROPANEDIOL | 5.4764 |
| A2 | SCLEROTIUM GUM | 0.3285 |
| A2 | HYALURONIC ACID | 0.0109 |
| A3 | POTASSIUM CETYL PHOSPHATE | 0.5476 |
| B | DIISOPROPYL SEBACATE | 10.9500 |
| B | [CETEARYL ALCOHOL; CETEARYL | 4.3811 |
| | GLUCOSIDE] | |

### Example 14

### Preparation of a Lipid Nanoparticle Composition Comprising the Emulsion of Example 13

The emulsion prepared in Example 13 is introduced into an appropriate recipient (phase A).

Separately, phase B (see Table 6) is prepared by dissolving N-Hance® CG17 Cationic Guar [INCI: GUAR HYDROXYPROPYLTRIMONIUM CHLORIDE; WATER (AQUA)] in water [INCI: WATER (AQUA)]. Phase B is added to phase A under intense stirring.

Components of phase C (Structure® XL [INCI: HYDROXYPROPYL STARCH PHOSPHATE] and Amigel® [INCI: SCLEROTIUM GUM]) and phase D (Heliogel™ [INCI: SODIUM ACRYLATES COPOLYMER; HYDROGENATED POLYISOBUTENE; LECITHIN; POLYGLYCERYL-10 STEARATE; SUNFLOWER (Helianthus annuus) SEED OIL; TOCOPHEROL]) are added slowly and one by one under intense stirring.

TABLE 6

| Phase | INGREDIENT | % weight |
|---|---|---|
| A | Emulsion of example 13 | q.s. 100% |
| B | [GUAR HYDROXYPROPYLTRIMONIUM | 0.20 |
| | CHLORIDE; WATER (AQUA)] | |
| B | WATER (AQUA) | 6.0 |
| C | HYDROXYPROPYL STARCH PHOSPHATE | 1.50 |
| C | SCLEROTIUM GUM | 0.75 |
| D | [SODIUM ACRYLATES COPOLYMER; | 0.25 |
| | HYDROGENATED POLYISOBUTENE; LECITHIN; | |
| | POLYGLYCERYL-10 STEARATE; SUNFLOWER | |
| | (HELIANTHUS ANNUUS) SEED OIL; | |
| | TOCOPHEROL] | |

### Example 15

Preparation of Liposomes Comprising Peptide H-Tyr-Val-Tyr-NH₂ (PEP-1-NH₂)

In an appropriate container, phase A is prepared by dissolving peptide H-Tyr-Val-Tyr-NH₂ (PEP-1-NH₂) in water [INCI: WATER (AQUA)]. Zemea™ [INCI: PROPANEDIOL] and 2-phenoxyethanol [INCI: PHENOXYETHANOL] (phase B) are added to phase A.

When all the previous components are dissolved, lecithin [INCI: LECITHIN] (phase C) is added little by little and under intense stirring, until complete dispersion. The finally obtained composition is shown in Table 7.

TABLE 7

| Phase | INGREDIENT | % weight |
|---|---|---|
| A | WATER (AQUA) | q.s. 100% |
| A | H-Tyr-Val-Tyr-NH₂ (PEP-1-NH₂) | 0.0526 |
| B | PROPANEDIOL | 8.5000 |
| B | PHENOXYETHANOL | 0.9050 |
| C | LECITHIN | 0.5000 |

The sample is homogenized with a titanium probe for 30 seconds.

### Example 16

### Preparation of Liposomes of Example 15 Bound to Cationic Polymers

The liposomes obtained in example 15 are added to SENSOMER™ CI-50 Polymer [INCI: WATER (AQUA), STARCH HYDROXYPROPYLTRIMONIUM CHLORIDE, UREA, SODIUM LACTATE, SODIUM CHLORIDE, SODIUM BENZOATE] at a liposomes:cationic polymer ratio of 95:5 (w/w) under slow stirring.

### Example 17

Preparation of a Cosmetic Composition (Cream) Containing H-Tyr-Val-Tyr-NH₂ (PEP-1-NH₂)

In a suitable vessel, the ingredients of phase A (water [INCI: WATER], Zemea™ [INCI: PROPANEDIOL], disodium EDTA [INCI: DISODIUM EDTA], and potassium sorbate [INCI: POTASSIUM SORBATE]) are dissolved under rotor stirring.

Next, phase A1 (Carbopol® Ultrez 10 Polymer [INCI: CARBOMER]) is added and let wet and disperse in the mixture. Phase A2 (Cola®Fax CPE-K [INCI: POTASSIUM CETYL PHOSPHATE]) is subsequently added and let to disperse too. The mixture is then heated at 70-75° C.

In a separate vessel, phase B ingredients (Schercemol™ DIA Ester [INCI: DIISOPROPYL ADIPATE], Phytocream 2000® [INCI: GLYCERYL STEARATE, CETEARYL ALCOHOL; POTASSIUM PALMITOYL HYDROLYZED WHEAT PROTEIN], 2-ethylhexyl cocoate [INCI: ETHYLHEXYL COCOATE], vitamin E acetate [INCI: TOCOPHERYL ACETATE], and 2-phenoxyethanol [INCI: PHENOXYETHANOL]) are weighed and the mixture is heated at 70-75° C.

When both mixtures reach the corresponding temperature, the emulsion is made by adding slowly phase B on the mixture of phases A under stirring with turbine.

When the mixture is cooling to 40° C., phases C₁ (glycerine [INCI: GLYCERIN], water [INCI: WATER], and H-Tyr-Val-Tyr-NH₂ (PEP-1-NH₂)]), and C₂ (dimethicone [INCI: DIMETHICONE]) followed by phase D (Novemer™ EC-2 Polymer [INCI: WATER; SODIUM ACRYLATES/BEHENETH-25 METACRYLATE CROSSPOLYMER; HYDROGENATED POLYDECENE; LAURYL GLUCOSIDE]) are sequentially added to it.

Last, phase E (perfume [INCI: FRAGANCE]) is added and phase F (water [INCI: WATER] and sodium hydroxide [INCI: SODIUM HYDROXIDE]) is then incorporated into the previous mixture for pH adjustment to 6.0-6.5.

The whole list of ingredients can be found in Table 8.

TABLE 8

| | | % |
|---|---|---|
| Phase | INGREDIENT | weight |
| A | WATER | 72.0000 |
| A | PROPANEDIOL | 10.0000 |
| A | DISODIUM EDTA | 0.2000 |
| A | POTASSIUM SORBATE | 0.1000 |
| A1 | CARBOMER | 0.5000 |
| A2 | POTASSIUM CETYL PHOSPHATE | 0.5000 |
| B | DIISOPROPYL ADIPATE | 5.0000 |
| B | [GLYCERYL STEARATE; CETEARYL ALCOHOL; | 5.0000 |
| | POTASSIUM PALMITOYL HYDROLYZED WHEAT | |
| | PROTEIN] | |
| B | ETHYLHEXYL COCOATE | 2.5000 |
| B | TOCOPHERYL ACETATE | 0.5000 |
| B | PHENOXYETHANOL | 0.5000 |
| C1 | GLYCERIN | 0.8000 |
| C1 | WATER | 0.1995 |
| C1 | H-Tyr-Val-Tyr-NH2 (PEP-1-NH₂) | 0.0005 |
| C2 | DIMETHICONE | 1.0000 |
| D | [WATER; SODIUM ACRYLATES/BEHENETH-25 | 1.0000 |
| | METACRYLATE CROSSPOLYMER; | |
| | HYDROGENATED POLYDECENE; LAURYL | |
| | GLUCOSIDE | |
| E | FRAGRANCE | 0.2000 |
| F | [WATER (AQUA); SODIUM HYDROXIDE] | q.s. |

### Example 18

Preparation of a Cosmetic Composition (Cream) Containing H-Tyr-Val-Tyr-NH₂ (PEP-1-NH₂)

In a suitable vessel, the ingredients of phase A (water [INCI: WATER], Zemea™ [INCI: PROPANEDIOL], disodium EDTA [INCI: DISODIUM EDTA], and potassium sorbate [INCI: POTASSIUM SORBATE]) are dissolved under rotor stirring.

Next, phase A1 (Carbopol® Ultrez 10 Polymer [INCI: CARBOMER]) is added and let wet and disperse in the mixture. Phase A2 (Cola®Fax CPE-K [INCI: POTASSIUM CETYL PHOSPHATE]) is subsequently added and let to disperse too. The mixture is then heated at 70-75° C.

In a separate vessel, phase B ingredients (Schercemol™ DIA Ester [INCI: DIISOPROPYL ADIPATE], Phytocream 2000® [INCI: GLYCERYL STEARATE, CETEARYL ALCOHOL; POTASSIUM PALMITOYL HYDROLYZED WHEAT PROTEIN], 2-ethylhexyl cocoate [INCI: ETHYLHEXYL COCOATE], vitamin E acetate [INCI: TOCOPHERYL ACETATE], and 2-phenoxyethanol [INCI: PHENOXYETHANOL]) are weighed and the mixture is heated at 70-75° C.

When both mixtures reach the corresponding temperature, the emulsion is made by adding slowly phase B on the mixture of phases A under stirring with a turbine.

When the mixture is cooling to 40° C., phases C₁ (glycerine [INCI: GLYCERIN], water [INCI: WATER], and H-Tyr-Val-Tyr-NH₂ (PEP-1-NH₂)]), and C₂ (dimethicone [INCI: DIMETHICONE]) followed by phase D (Novemer™ EC-2 Polymer [INCI: WATER; SODIUM ACRYLATES/BEHENETH-25 METACRYLATE CROSSPOLYMER; HYDROGENATED POLYDECENE; LAURYL GLUCOSIDE]) are sequentially added to it.

Last, phase E (perfume [INCI: FRAGANCE]) is added and phase F (water [INCI: WATER] and sodium hydroxide [INCI: SODIUM HYDROXIDE]) is then incorporated into the previous mixture for pH adjustment to 6.0-6.5.

The whole list of ingredients can be found in Table 9.

TABLE 9

| | | % |
|---|---|---|
| Phase | INGREDIENT | weight |
| A | WATER | 71.000 |
| A | PROPANEDIOL | 10.000 |
| A | DISODIUM EDTA | 0.200 |
| A | POTASSIUM SORBATE | 0.100 |
| A1 | CARBOMER | 0.500 |
| A2 | POTASSIUM CETYL PHOSPHATE | 0.500 |
| B | DIISOPROPYL ADIPATE | 5.000 |
| B | [GLYCERYL STEARATE; CETEARYL ALCOHOL; | 5.000 |
| | POTASSIUM PALMITOYL HYDROLYZED WHEAT | |
| | PROTEIN] | |
| B | ETHYLHEXYL COCOATE | 2.500 |
| B | TOCOPHERYL ACETATE | 0.500 |
| B | PHENOXYETHANOL | 0.500 |
| C1 | GLYCERIN | 1.600 |
| C1 | WATER | 0.399 |
| C1 | H-Tyr-Val-Tyr-NH2 (PEP-1-NH₂) | 0.001 |
| C2 | DIMETHICONE | 1.000 |
| D | [WATER; SODIUM ACRYLATES/BEHENETH-25 | 1.000 |
| | METACRYLATE CROSSPOLYMER; | |
| | HYDROGENATED POLYDECENE; LAURYL | |
| | GLUCOSIDE | |
| E | FRAGRANCE | 0.200 |
| F | [WATER (AQUA); SODIUM HYDROXIDE] | q.s. |

### Example 19

Preparation of a Cosmetic Composition (Cream) Containing H-Tyr-Val-Tyr-NH₂ (PEP-1-NH₂)

In a suitable vessel, the ingredients of phase A (water [INCI: WATER], Microcare® Emollient PTG [INCI: PENTYLENE GLYCOL], glycerine [INCI: GLYCERIN], GENENCARE OSMS BA™ [INCI: BETAINE], Microcare® BNA [INCI: BENZYL ALCOHOL] and 2-phenoxyethanol [INCI: PHENOXYETHANOL]) are dissolved under rotor stirring.

Next, phase A1 (Carbopol® Ultrez 10 Polymer [INCI: CARBOMER]) is added and let wet and disperse in the mixture. Phase A2 (Cola®Fax CPE-K [INCI: POTASSIUM CETYL PHOSPHATE]) is subsequently added and let to disperse too. The mixture is then heated at 70-75° C.

In a separate vessel, phase B ingredients (Massocare® HD [INCI: ISOHEXADECANE], Lincol Bas™ [INCI: C12-15 ALKYL BENZOATE], Gandak C™ [INCI: CETYL ALCOHOL], Sorbital™ T20P [INCI: POLYSORBATE-20], Vegetable stearic acid 50/50 [INCI: STEARIC ACID, PALMITIC ACID]) are weighed and the mixture is heated at 70-75° C.

When both mixtures reach the corresponding temperature, the emulsion is made by adding slowly phase B on the mixture of phases A under stirring with turbine.

When the mixture is cooling to 40° C., phases C (Cyclomethicone [INCI: CYCLOMETHICONE] and (Zemea™ [INCI: PROPANEDIOL], and H-Tyr-Val-Tyr-NH₂ (PEP-1-NH₂)])) are sequentially added to it.

Last, phase D (perfume [INCI: FRAGANCE]) is added and phase E (water [INCI: WATER] and sodium hydroxide [INCI: SODIUM HYDROXIDE]) is then incorporated into the previous mixture for pH adjustment to 6.0-6.5.

The whole list of ingredients can be found in Table 10.

TABLE 10

| Phase | INGREDIENT | % weight |
|---|---|---|
| A | WATER | 76.150 |
| A | PENTYLENE GLYCOL | 5.000 |
| A | GLYCERIN | 3.000 |
| A | BETAINE | 3.000 |
| A | BENZYL ALCOHOL | 0.400 |
| A | PHENOXYETHANOL | 0.500 |
| A1 | CARBOMER | 0.350 |
| A2 | POTASSIUM CETYL PHOSPHATE | 0.400 |
| B | ISOHEXADECANE | 2.000 |
| B | C12-15 ALKYL BENZOATE | 2.000 |
| B | CETYL ALCOHOL | 1.800 |
| B | POLYSORBATE-20 | 0.800 |
| B | [STEARIC ACID, PALMITIC ACID] | 0.500 |
| C | PROPANEDIOL | 1.990 |
| C | H-Tyr-Val-Tyr-NH2 (PEP-1-NH₂) | 0.010 |
| C | CYCLOMETHICONE | 2.000 |
| E | FRAGRANCE | 0.200 |
| F | [WATER (AQUA); SODIUM HYDROXIDE] | q.s. |

### Example 20

### In Vivo Study in Caucasian Skin Type Female Volunteers.

Skin aging is a complex biological process influenced by a combination of intrinsic and extrinsic factors. Skin aging is associated with connective tissue weakness (i.e., loss of firmness and elasticity). Moreover, age-related connective tissue weakness in the extremities can lead to unsightly cosmetic skin defect referred to as cellulite. This study is designed to evaluate the improvement of skin appearance, particularly in relation with cellulite. The study is carried out during 8 weeks for evaluating the in vivo benefits of the compositions of the invention. 20 Caucasian female volunteers are included. Subjects apply the composition described in Example 19 (ACTIVE CREAM) on one buttock (left or right) and a PLACEBO cream on the other buttock. PLACEBO has the same composition of ACTIVE CREAM, but without the peptide of the invention. ACTIVE CREAM and PLACEBO are applied for 8 weeks twice a day (morning and night). The subjects serve as their own reference and results obtained at time 8 weeks are compared with those obtained at initial time. Moreover, results obtained with the ACTIVE CREAM are compared with those obtained with PLACEBO CREAM.

The improvement in skin appearance of the product is assessed by Firmness and Elasticity Assessment as well as Self-Questionnaires.

### Firmness and Elasticity Assessment:

Firmness and elasticity measurements are taken with a Cutometer® device (Courage & Khazaka, Koln, Germany) in each buttock at baseline and after 8 weeks of product application.

Table 11 shows the increase of firmness and elasticity of skin after 8 weeks of product application, where:
R0 parameter refers to the maximum amplitude after applying suction on the skin using a negative pressure. The higher the resistance of the skin to negative pressure the firmer the skin. Decrease of R0 parameter means an increase of skin firmness.
R5 parameter refers to net elasticity after applying suction on the skin using a negative pressure. An increase of R5 parameter means an increase of skin elasticity.

TABLE 11

| | R0 | R5 |
|---|---|---|
| Active Cream (%) | −1.69 | 4.78 |
| Placebo Cream (%) | 1.99 | −2.77 |

Results demonstrate that, after 8 weeks of application of ACTIVE CREAM, there is an increase of skin firmness compared to PLACEBO and versus initial time. Moreover, there is an increase of skin elasticity compared to PLACEBO and versus initial time.

### Self-Questionnaire:

After 8 weeks of product application of ACTIVE CREAM and PLACEBO volunteers answered a self-questionnaire to evaluate the efficacy of both products.

The percentages of positive responses to skin and cellulite-related questions after 8 weeks of product application are summarized in Table 12:

TABLE 12

| | % of positive | % of positive |
|---|---|---|
| | responses | responses |
| | Active cream | Placebo cream |
| There is a decrease in the | 70.83 | 50.00 |
| appearance of cellulite | | |
| The orange peel skin is less | 70.83 | 58.33 |
| visible | | |
| There is a reduction in the | 41.67 | 37.50 |
| presence of nodules in the skin | | |
| The skin is firmer | 62.50 | 45.83 |
| There is an improvement on the | 75.00 | 70.83 |
| skin overall appearance | | |

Results demonstrate that, after 8 weeks of application, the ACTIVE CREAM has more efficacy than PLACEBO, according to the answers of volunteers.

In conclusion, the composition of the invention is shown to have the capacity to increase skin firmness and elasticity and thus improve the overall appearance of the skin. Enhancement of connective tissue firmness can improve skin texture and structure, which also results in a decrease of the appearance of cellulite and orange peel skin.

The Sequence Listing, submitted herewith, is incorporated herein in its entirety, by reference.

1. A compound of formula (I):
R₁—Wₘ—Xₙ-AA₁-AA₂-AA₃-Yₚ—Z_{q}—R₂ (I),
its stereoisomers and/or its cosmetically or pharmaceutically acceptable salts, wherein:
AA₁ is selected from the group consisting of Tyr and Trp;
AA₂ is selected from the group consisting of Val, Ile, Leu and;
AA₃ is selected from the group consisting of Tyr, Phe and Trp;
W, X, Y and Z are each independently an amino acid;
m, n, p and q are each independently 0 or 1;
m+n+p+q is less than or equal to 2;
R₁ is selected from the group consisting of H, a polymer derived from polyethylene glycol, a non-cyclic aliphatic group, alicyclyl, heterocyclyl, heteroarylalkyl, aryl, aralkyl and R₅—CO—, wherein R₅ is selected from the group consisting of H, a non-cyclic aliphatic group, alicyclyl, aryl, aralkyl, heterocyclyl and heteroarylalkyl;
R₂ is selected from the group consisting of —NR₃R₄, —OR₃, and —SR₃, wherein R₃ and R₄ are independently selected from a group consisting of H, a polymer derived from polyethylene glycol, a non-cyclic aliphatic group, alicyclyl, heterocyclyl, heteroarylalkyl, aryl and aralkyl;
wherein when AA₂ is Leu, AA₁ is Tyr, and AA₃ is Tyr; and
R₁ and R₂ are not amino acids.

2. The compound according to claim 1, wherein R₁ is selected from the group consisting of H and R₅—CO—, wherein R₅ is selected from the group consisting of C₁-C₁₈ alkyl, C₂-C₂₄ alkenyl, C₃-C₂₄ cycloalkyl; and R₂ is —NR₃R₄ or —OR₃ wherein R₃ and R₄ are independently selected from the group consisting of H and C₁-C₁₆ alkyl.

3. The compound according to claim 2, wherein R₁ is selected from the group consisting of H, acetyl, tert-butanoyl, prenyl, hexanoyl, 2-methylhexanoyl, cyclohexanecarboxyl, octanoyl, decanoyl, lauroyl, myristoyl, palmitoyl, stearoyl, oleoyl and linoleoyl.

4. The compound according to claim 2, wherein R₁ is selected from the group consisting of H and R₅—CO—, where R₅ is selected from the group consisting of C₁-C₁₆ alkyl and C₂-C₁₈ alkenyl.

5. The compound according to claim 2, wherein R₂ is —NR₃R₄ or —OR₃, where R₃ is H_{A} and R₄ is selected from the group consisting of H and C₁-C₁₆ alkyl.

6. The compound according to claim 1, having the formula H-Tyr-Val-Tyr-NH₂.

7. (canceled)

8. (canceled)

9. (canceled)

10. (canceled)

11. A cosmetic or pharmaceutical composition comprising a cosmetically effective or pharmaceutically effective quantity of a compound of formula (I), its stereoisomers and/or its cosmetically or pharmaceutically acceptable salts, according to claim 1, and at least one cosmetically acceptable excipient or adjuvant.

12. The composition according to claim 11, wherein the compound of formula (I), its stereoisomers and/or its cosmetically or pharmaceutically acceptable salts, is incorporated into a cosmetically or pharmaceutically acceptable delivery system or sustained release system selected from the group consisting of liposomes, mixed liposomes, oleosomes, niosomes, ethosomes, milliparticles, microparticles, nanoparticles, solid lipid nanoparticles, nanostructured lipid carriers, sponges, cyclodextrins, vesicles, micelles, mixed micelles of surfactants, surfactant-phospholipid mixed micelles, millispheres, microspheres, nanospheres, lipospheres, millicapsules, microcapsules, nanocapsules, microemulsions and nanoemulsions, or is adsorbed on a solid organic polymer or solid mineral support selected from the group consisting of talc, bentonite, silica, starch and maltodextrin.

13. The composition according to claim 11, wherein the composition is in a formulation selected from the group consisting of creams, multiple emulsions, anhydrous compositions, aqueous dispersions, oils, milks, balsams, foams, lotions, gels, cream gels, hydroalcoholic solutions, hydroglycolic solutions, hydrogels, liniments, sera, soaps, shampoos, conditioners, serums, polysaccharide films, ointments, mousses, pomades, powders, bars, pencils, sprays, aerosols, capsules, gelatin capsules, soft capsules, hard capsules, tablets, sugar coated tablets, pills, powders, granules, chewing gum, solutions, suspensions, emulsions, syrups, elixirs, jellies and gelatins.

14. A cosmetic, non-therapeutic method of treatment and/or care of the skin, hair, nails and/or mucous membranes in a subject comprising administering a cosmetically effective amount of a compound according to claim 1, its stereoisomers and/or its cosmetically acceptable salts or a cosmetic composition comprising a cosmetically effective amount of said compound, its stereoisomers and/or its cosmetically acceptable salts, to the skin, hair, nails and/or mucous membrane of the subject.

15. The cosmetic, non-therapeutic method according to claim 14, wherein the treatment and/or care is: the reduction of lipid accumulation in the skin; the stimulation of lipolysis in the skin; the treatment of cellulite; the treatment of aging of the skin; the treatment of eye bags; the treatment of skin wrinkles; maintaining and/or improving skin firmness; maintaining and/or improving skin elasticity; the promotion or stimulation of collagen synthesis; face and neck modelling; and/or reducing a BMI value of the subject.

16. The compound according to claim 4, wherein R₁ is selected from the group consisting of H, acetyl, myristoyl and palmitoyl.

17. The compound according to claim 5, wherein R₂ is selected from the group consisting of —OH, —NH₂ and —NHR₄, where R₄ is C₁-C₁₆ alkyl.

18. The compound according to claim 1, wherein AA₁ is Tyr, AA₂ is selected from the group consisting of Val, Ile, and Met, and AA₃ is selected from the group consisting of Tyr, Phe, and Trp.

19. The compound according to claim 1, wherein the formula (I) is selected from H-Tyr-Val-Tyr-NH₂; H-Tyr-Val-Tyr-OH; H-Tyr-Val-Tyr-NHC₆H₁₃; H-Tyr-Val-Tyr-NHC₁₆H₃₃; H-Tyr-Val-Tyr-Phe-NH₂; H-Tyr-Val-Tyr-Trp-NH₂; H-Phe-Tyr-Val-Tyr-Phe-NH₂; H-Phe-Trp-Tyr-Val-Tyr-NH₂; H-Tyr-Val-Tyr-Phe-Trp-NH₂; Ac-Tyr-Val-Tyr-NH₂; Myr-Tyr-Val-Tyr-NH₂; and Palm-Tyr-Val-Tyr-NH₂.

20. A method of treatment and/or care of the skin, hair, nails and/or mucous membranes of a subject comprising administering, to the skin, hair, nails and/or mucous membranes of the subject, an effective amount of a compound R₁—Wₘ—Xₙ-AA₁-AA₂-AA₃-Yₚ—Z_{q}—R₂ (I), its stereoisomers and/or its cosmetically or pharmaceutically acceptable salts, or a composition containing the compound, its stereoisomers and/or its cosmetically or pharmaceutically acceptable salts, wherein:
AA₁ is selected from the group consisting of Tyr and Trp;
AA₂ is selected from the group consisting of Val, Ile, Leu, and Met;
AA₃ is selected from the group consisting of Tyr, Phe and Trp;
W, X, Y and Z are each independently an amino acid;
m, n, p and q are each independently 0 or 1;
m+n+p+q is less than or equal to 2;
R₁ is selected from the group consisting of H, a polymer derived from polyethylene glycol, a non-cyclic aliphatic group, alicyclyl, heterocyclyl, heteroarylalkyl, aryl, aralkyl and R₅—CO—, wherein R₅ is selected from the group consisting of H, a non-cyclic aliphatic group, alicyclyl, aryl, aralkyl, heterocyclyl and heteroarylalkyl;
R₂ is selected from the group consisting of —NR₃R₄, —OR₃, and —SR₃, wherein R₃ and R₄ are independently selected from a group consisting of H, a polymer derived from polyethylene glycol, a non-cyclic aliphatic group, alicyclyl, heterocyclyl, heteroarylalkyl, aryl and aralkyl; and
R₁ and R₂ are not amino acids.

21. The method according to claim 20, wherein the treatment and/or care comprises at least one of: the reduction of lipid accumulation in the skin; the stimulation of lipolysis in the skin; the treatment of cellulite; the treatment of aging of the skin; the treatment of eye bags; maintaining and/or improving skin firmness; maintaining and/or improving skin elasticity; the treatment of skin wrinkles; the promotion or stimulation of collagen synthesis; face and neck modelling; and reducing a BMI value of the subject.

22. The method according to claim 20, wherein the treatment and/or care comprises the treatment of inflammation of the skin of the subject.

23. The method according to claim 20, wherein the treatment and/or care comprises modulating nocturnin in the subject.


SEQUENCE LISTING (10 sequences)

SEQ ID NO: 1 - protein, 4 aa, Artificial Sequence
  peptide
  Xaa (1)..(1): Xaa is Tyr or Trp
  Xaa (2)..(2): Xaa is Val, Ile, Leu or Met
  Xaa (3)..(3): Xaa is Tyr, Phe or Trp
  Xaa (4)..(4): Xaa is any amino acid
XXXX

SEQ ID NO: 2 - protein, 4 aa, Artificial Sequence
  peptide
  Xaa (1)..(1): Xaa is any amino acid
  Xaa (2)..(2): Xaa is Tyr or Trp
  Xaa (3)..(3): Xaa is Val, Ile, Leu or Met
  Xaa (4)..(4): Xaa is Tyr, Phe or Trp
XXXX

SEQ ID NO: 3 - protein, 5 aa, Artificial Sequence
  peptide
  Xaa (1)..(1): Xaa is Tyr or Trp
  Xaa (2)..(2): Xaa is Val, Ile, Leu or Met
  Xaa (3)..(3): Xaa is Tyr, Phe or Trp
  Xaa (4)..(4): Xaa is any amino acid
  Xaa (5)..(5): Xaa is any amino acid
XXXXX

SEQ ID NO: 4 - protein, 5 aa, Artificial Sequence
  No comment
  Xaa (1)..(1): Xaa is any amino acid
  Xaa (2)..(2): Xaa is any amino acid
  Xaa (3)..(3): Xaa is Tyr or Trp
  Xaa (4)..(4): Xaa is Val, Ile, Leu or Met
  Xaa (5)..(5): Xaa is Tyr, Phe or Trp
XXXXX

SEQ ID NO: 5 - protein, 5 aa, Artificial Sequence
  peptide
  Xaa (1)..(1): Xaa is any amino acid
  Xaa (2)..(2): Xaa is Tyr or Trp
  Xaa (3)..(3): Xaa is Val, Ile, leu or Met
  Xaa (4)..(4): Xaa is Tyr, Phe or Trp
  Xaa (5)..(5): Xaa is any amino acid
XXXXX

SEQ ID NO: 6 - protein, 4 aa, Artificial Sequence
  peptide
YVYF

SEQ ID NO: 7 - protein, 4 aa, Artificial Sequence
  peptide
YVYW

SEQ ID NO: 8 - protein, 5 aa, Artificial Sequence
  peptide
FYVYF

SEQ ID NO: 9 - protein, 5 aa, Artificial Sequence
  peptide
FWYVY

SEQ ID NO: 10 - protein, 5 aa, Artificial Sequence
  peptide
YVYFW